(19) United States
(12) Patent Application Publication
(10) Pub. No.: US 2023/0150936 A1
(43) Pub. Date: May 18, 2023

(54) ANALOGS OF N,N,N-TRIMETHYL-4-PHOSPHORYLOXYTRYPTAMINE

(71) Applicant: Terran Biosciences, Inc., New York, NY (US)

(72) Inventors: Matthew DUNCTON, San Bruno, CA (US); Samuel CLARK, New York, NY (US)

(21) Appl. No.: 17/982,451

(22) Filed: Nov. 7, 2022

Related U.S. Application Data
(60) Provisional application No. 63/276,279, filed on Nov. 5, 2021.

(51) Int. Cl. C07D 209/16

(52) U.S. Cl. CPC C07D 209/16; C07B 2200/05

(57) ABSTRACT

Deuterium-containing analogs/derivatives of N,N,N-trimethyl phosphoryloxytryptamine are disclosed. The deuterated compounds can be incorporated into pharmaceutical compositions and used to treat diseases such as psychological diseases and disorders.

### CROSS REFERENCE TO RELATED APPLICATIONS

This application claims priority to and the benefit of U.S. Provisional Patent Application No. 63/276,279, filed Nov. 5, 2021. The contents are incorporated by reference in its entirety for all purposes.

### BACKGROUND

Aeruginascin, N,N,N-trimethyl-4-phosphoryloxytryptamine, is a derivative of psilocybin and exhibits novel pharmacological properties. It is believed that aeruginascin undergoes hydrolysis in vivo to generate its active metabolite 4-hydroxy-N,N,N-trimethyltryptamine.

### SUMMARY

According to the present invention, a compound has the structure encompassed by Formula (2),

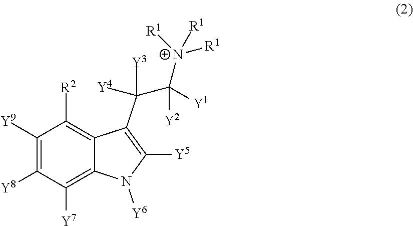

or a pharmaceutically acceptable salt thereof, wherein,

each R¹ is independently selected from CH₃, CH₂D, CHD₂, and CD₃;

R² is selected from an oxygen radical, a phosphate radical, a sulfate radical, and the moiety —O—C(═O)—R³ where R³ is selected from CH₃, CH₂D, CHD₂, and CD₃;

each of Y¹, Y², Y³, Y⁴, Y⁵, Y⁶, Y⁷, Y⁸, and Y⁹ is independently selected from hydrogen and deuterium; and

one or more of R¹, Y¹, Y², Y³, Y⁴, Y⁵, Y⁶, Y⁷, Y⁸, and Y⁹ comprises deuterium.

According to the present invention, pharmaceutical compositions comprise a compound according to the present invention, or a pharmaceutically acceptable salt thereof.

According to the present invention, methods treating a disease in a patient comprise administering to a patient in need of such treatment a therapeutically effective amount of a compound according to the present invention, or a pharmaceutically acceptable salt thereof or a pharmaceutical composition according to the present invention, wherein the disease is selected from a psychological disease.

### DETAILED DESCRIPTION

A dash (“—”) that is not between two letters or symbols is used to indicate a point of attachment for a moiety or substituent. For example, —CONH₂ is attached through the carbon atom.

“Compounds provided by the present disclosure” include compounds of Formula (2), enantiomers of compounds of Formula (2), pharmaceutically acceptable salt of any of the foregoing, hydrates of any of the foregoing, and solvates of any of the foregoing.

Compounds and moieties disclosed herein include optical isomers of compounds and moieties, racemates thereof, and other mixtures thereof. In such embodiments, the single enantiomers or diastereomers may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column with chiral stationary phases. In addition, compounds include (Z)- and (E)-forms (or cis- and trans-forms) of compounds with double bonds either as single geometric isomers or mixtures thereof.

Compounds and moieties may also exist in several tautomeric forms including the enol form, the keto form, and mixtures thereof. Accordingly, the chemical structures depicted herein encompass all possible tautomeric forms of the illustrated compounds. Compounds may exist in unsolvated forms as well as solvated forms, including hydrated forms. Certain compounds may exist in multiple crystalline, co-crystalline, or amorphous forms. Compounds include pharmaceutically acceptable salts thereof, or pharmaceutically acceptable solvates of the free acid form of any of the foregoing, as well as crystalline forms of any of the foregoing.

“Disease” refers to a disease, disorder, condition, or symptom of any of the foregoing.

“Drug” as defined under 21 U.S.C. § 321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . . ”.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids and one or more protonable functional groups such as primary, secondary, or tertiary amines within the parent compound. Examples of suitable inorganic acids include hydrochloric acid, hydrobromic acid, sulfuric acid, nitric acid, phosphoric acid, and the like. A salt can be formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. A salt can be formed when one or more acidic protons present in the parent compound are replaced by a metal ion, e.g., an alkali metal ion, an alkaline earth ion, or an aluminum ion, or combinations thereof; or coordinates with an organic base such as ethanolamine, diethanolamine, triethanolamine, N-methylglucamine, and the like. A pharmaceutically acceptable salt can be the hydrochloride salt. A pharmaceutically acceptable salt can be the sodium salt. In compounds having two or more ionizable groups, a pharmaceutically acceptable salt can comprise one or more counterions, such as a bi-salt, for example, a dihydrochloride salt.

The term “pharmaceutically acceptable salt” includes hydrates and other solvates, as well as salts in crystalline or non-crystalline form. Where a particular pharmaceutically acceptable salt is disclosed, it is understood that the particular salt (e.g., a hydrochloride salt) is an example of a salt, and that other salts may be formed using techniques known to one of skill in the art. Additionally, one of skill in the art would be able to convert the pharmaceutically acceptable salt to the corresponding compound, free base and/or free acid, using techniques generally known in the art.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a compound of Formula (2) or a pharmaceutically acceptable salt thereof and at least one pharmaceutically acceptable vehicle, with which the compound of Formula (2) or a pharmaceutically acceptable salt thereof is administered to a patient. Pharmaceutically acceptable vehicles are known in the art.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). In some embodiments, “preventing” or “prevention” refers to reducing symptoms of the disease by administering a compound provided by the present disclosure in a preventative fashion. The application of a therapeutic agent for preventing or prevention of a disease of disorder is known as ‘prophylaxis.’ Compounds provided by the present disclosure can provide superior prophylaxis because of lower long-term side effects over long time periods.

“Solvate” refers to a molecular complex of a compound with one or more solvent molecules in a stoichiometric or non-stoichiometric amount. Such solvent molecules are those commonly used in the pharmaceutical arts, which are known to be innocuous to a patient, such as water or ethanol. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Solvates” refers to incorporation of solvents into to the crystal lattice of a compound described herein, in stoichiometric proportions, resulting in the formation of an adduct. Methods of making solvates include, for example, storage in an atmosphere containing a solvent, dosage forms that include the solvent, or routine pharmaceutical processing steps such as, for example, crystallization (i.e., from solvent or mixed solvents) vapor diffusion. Solvates may also be formed, under certain circumstances, from other crystalline solvates or hydrates upon exposure to the solvent or upon suspension material in solvent. Solvates may crystallize in more than one form resulting in solvate polymorphism.

“Treating” or “treatment” of a disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the patient. “Treating” or “treatment” also refers to delaying the onset of the disease or delaying the onset of at least one or more symptoms thereof in a patient who may be exposed to or predisposed to a disease or disorder even though that patient does not yet experience or display symptoms of the disease.

“Therapeutically effective amount” refers to the amount of a compound that, when administered to a patient for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

Reference is now made to certain compounds and methods. The disclosed embodiments are not intended to be limiting of the claims. To the contrary, the claims are intended to cover all alternatives, modifications, and equivalents.

Compounds provided by the present disclosure are deuterated analogs and derivatives of N,N,N-trimethyl-4-phosphoryloxytryptamine, 4-hydroxy-N,N,N-trimethyltryptamine (4-OH TMT) and 4-acetoxy-N,N,N-trimethyltryptamine iodide (4-OAc TMT). The deuterated analogs and derivatives exhibit improved pharmacological properties.

N,N,N-Trimethyl-4-phosphoryloxytryptamine, also known as aeruginascin, has the structure of Formula (1):

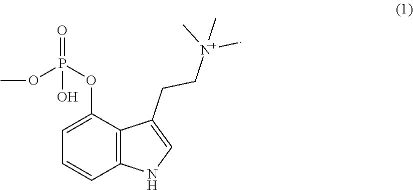

A compound provided by the present disclosure can have the structure of Formula (2),

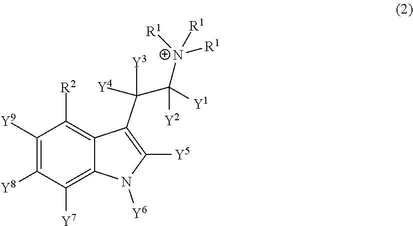

or a pharmaceutically acceptable salt thereof, wherein,

each R¹ can be independently selected from CH₃, CH₂D, CHD₂, and CD₃;

R² can be selected from an oxygen radical, a phosphate radical, a sulfate radical, and the moiety —O—C(═O)—R³ where R³ is selected from CH₃, CH₂D, CHD₂, and CD₃;

each of Y¹, Y², Y³, Y⁴, Y⁵, Y⁶, Y⁷, Y⁸, and Y⁹ can be independently selected from hydrogen and deuterium; and

one or more of R¹, Y¹, Y², Y³, Y⁴, Y⁵, Y⁶, Y⁷, Y⁸, and Y⁹ comprises deuterium.

A compound of Formula (2) can have the structure of Formula (2a), Formula (2b), Formula (2c), or Formula (2d):

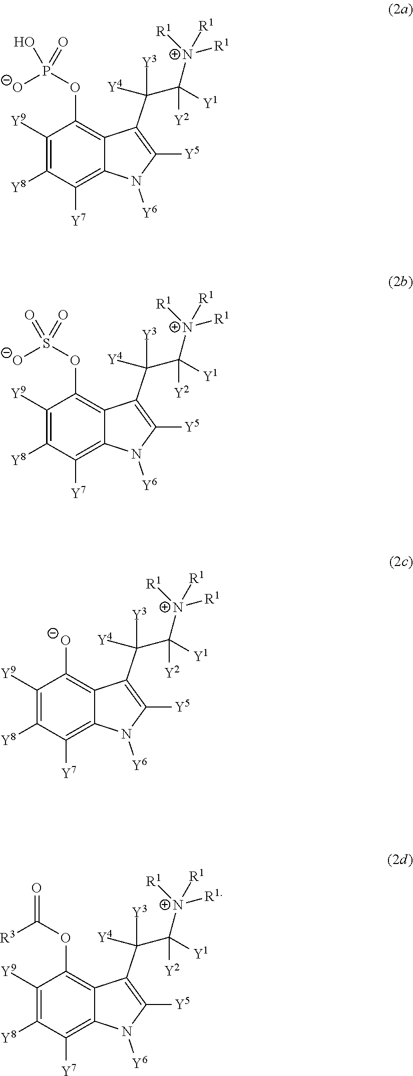

In a compound of Formula (2), the compound can have the structure of Formula (2a).

In a compound of Formula (2), the compound can have the structure of Formula (2b).

In a compound of Formula (2), the compound can have the structure of Formula (2c).

In a compound of Formula (2), the compound can have the structure of Formula (2d).

In a compound of Formula (2), R² can be an oxygen radical (—O).

In a compound of Formula (2), R² can be a phosphate radical (—OP(═O)(—OH)(—O″).

In a compound of Formula (2), R² can be a sulfate radical (—OS(═O)₂(—O).

In a compound of Formula (2), R² can be —O—C(═O)—R³.

In a compound of Formula (2), wherein R² is —O—C(═O)—R³, R³ can be CH₃.

In a compound of Formula (2), wherein R² is —O—C(═O)—R³, R³ can be CH₂D.

In a compound of Formula (2), wherein R² is —O—C(═O)—R³, R³ can be CHD₂.

In a compound of Formula (2), wherein R² is —O—C(═O)—R³, R³ can be CD₃.

In a compound of Formula (2), each of Y⁵, Y⁶, Y⁷, Y⁸, and Y⁹ can be hydrogen.

In a compound of Formula (2), each of Y⁵, Y⁶, Y⁷, Y⁸, and Y⁹ can be deuterium.

In a compound of Formula (2), one of Y⁵, Y⁶, Y⁷, Y⁸, and Y⁹ can be hydrogen and each of the other of Y⁵, Y⁶, Y⁷, Y⁸, and Y⁹ can be deuterium.

In a compound of Formula (2), two of Y⁵, Y⁶, Y⁷, Y⁸, and Y⁹ can be hydrogen and each of the other of Y⁵, Y⁶, Y⁷, Y⁸, and Y⁹ can be deuterium.

In a compound of Formula (2), three of Y⁵, Y⁶, Y⁷, Y⁸, and Y⁹ can be hydrogen and the other of Y⁵, Y⁶, Y⁷, Y⁸, and Y⁹ can be deuterium.

In a compound of Formula (2), four of Y⁵, Y⁶, Y⁷, Y⁸, and Y⁹ can be hydrogen and the other of Y⁵, Y⁶, Y⁷, Y⁸, and Y⁹ can be deuterium.

In a compound of Formula (2), each R¹ can independently be selected from CH₃, CH₂D, CHD₂, and CD₃.

In a compound of Formula (2), each R¹ can independently be selected from CH₃ and CD₃.

In a compound of Formula (2), each R¹ can be CH₃.

In a compound of Formula (2), each R¹ can be CD₃.

In a compound of Formula (2), each of Y′, Y², Y³, and Y⁴ can be deuterium.

In a compound of Formula (2), each of Y¹ and Y² can be deuterium, and each of Y³ and Y⁴ can be hydrogen.

In a compound of Formula (2), each of Y¹ and Y² can be hydrogen, and each of Y³ and Y⁴ can be deuterium.

In a compound of Formula (2), each of Y′, Y², and Y³ can be hydrogen, and Y⁴ can be deuterium.

In a compound of Formula (2), each of Y² and Y³ can be hydrogen, and each of Y¹ and Y⁴ can be deuterium.

In a compound of Formula (2), Y² can be hydrogen, and each of Y′, Y³ and Y⁴ can be deuterium.

In a compound of Formula (2), Y³ can be hydrogen, and each of Y′, Y² and Y⁴ can be deuterium.

In a compound of Formula (2), Y¹, Y², Y³, and Y⁴ can be hydrogen.

In a compound of Formula (2), the compound does not comprise compounds having the structure of Formula (3a)-3(i):

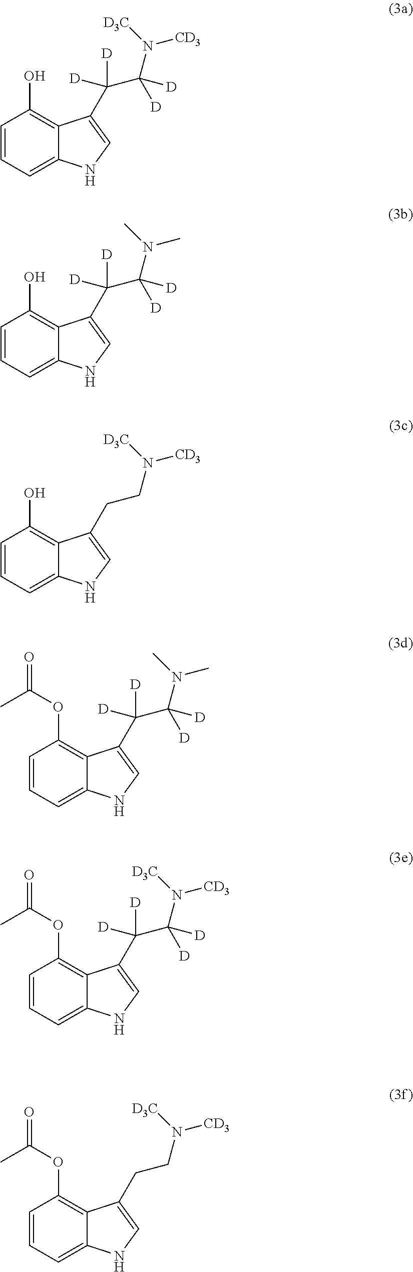

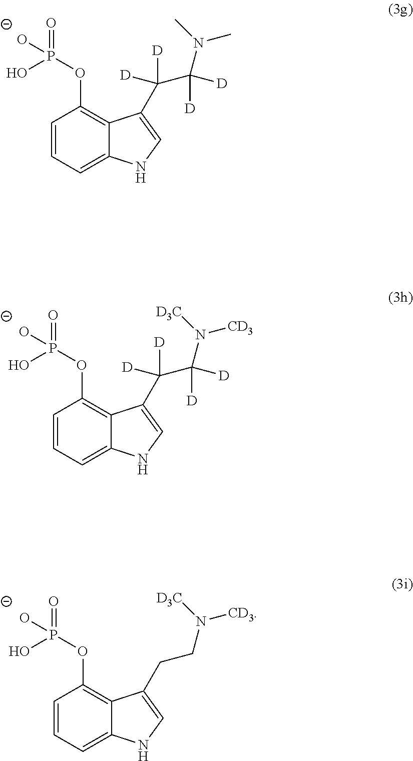 [SMILES: [2H]C([2H])(c1c[nH]c2cccc(OP(=O)([O-])O)c12)C([2H])([2H])N(C)C ; [2H]C([2H])([2H])N(C([2H])([2H])[2H])C([2H])([2H])C([2H])([2H])c1c[nH]c2cccc(OP(=O)(O)O)c12 ; [2H]C([2H])([2H])N(CCc1c[nH]c2cccc(OP(=O)(O)O)c12)C([2H])([2H])[2H]]

Compounds having the structure of Formula (3a)-(3i), which are not included within the scope of the present invention, correspond to compounds having the following chemical nomenclature:

3-(2-(bis(methyl-d3)amino)ethyl-1,1,2,2-d4)-1H-indol-4-ol (3 a);

3-(2-(dimethylamino)ethyl-1,1,2,2-d4)-1H-indol-4-ol (3b);

3-(2-(bis(methyl-d3)amino)ethyl)-1H-indol-4-ol (3c);

3-(2-(dimethylamino)ethyl-1,1,2,2-d4)-1H-indol-4-yl acetate (3d);

3-(2-(bis(methyl-d3)amino)ethyl-1,1,2,2-d4)-1H-indol-4-yl acetate (3 e);

3-(2-(bis(methyl-d3)amino)ethyl)-1H-indol-4-yl acetate (3f);

3-(2-(dimethylamino)ethyl-1,1,2,2-d4)-1H-indol-4-yl hydrogen phosphate (3g);

3-(2-(bis(methyl-d3)amino)ethyl-1,1,2,2-d4)-1H-indol-4-yl hydrogen phosphate (3h); and

3-(2-(bis(methyl-d3)amino)ethyl)-1H-indol-4-yl hydrogen phosphate (3i).

In a compound of Formula (2), R² can be selected from an oxygen radical, a phosphate radical, a sulfate radical, the salt can be selected from the hydrochloride (HCl) salt, the hydrogen bromine (HBr) salt, the hydrogen iodide (HI) salt, the sulfuric acd (H₂SO₄) salt, the hydrogen phosphate (H₃PO₄) salt, the paratoluene sulfonic acid (pTsOH) salt, the mesylate (MsOH) salt, and the phenylacetic acid (PhCo2H) salt.

In a compound of Formula (2), R² can be —O—C(═O)—R³, and the salt can be selected from the chloride salt, iodine salt, the bromine salt, the sulfate salt the phosphate salt, the paratolunne sulfonic acid salt, the mesylate salt, and the phenylacetic acid salt.

A compound of Formula (2) can be a pharmaceutically acceptable salt of a compound of Formula (2), a hydrate thereof, or a solvate of any of the foregoing.

Compounds of Formula (2) can be synthesized using methods known in the art.

The following general chemistry for the synthesis of non-deuterated analogs of compounds of Formula (2) is described in Chadeayne et al, ACS Omega 2020, 5, 16940-16943 according to reaction Scheme (1):

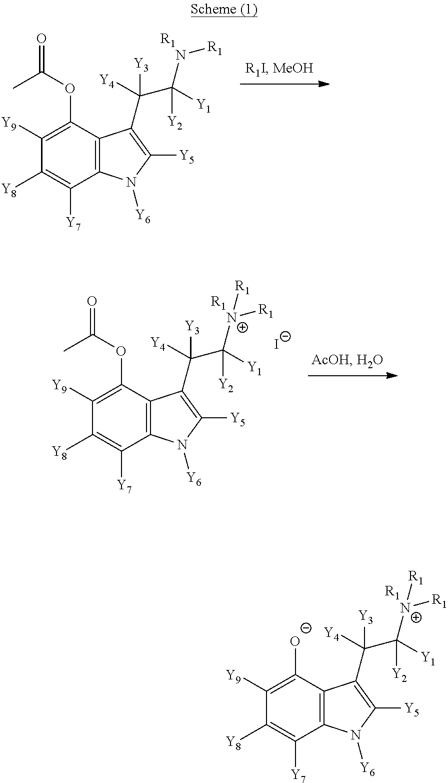

The following general chemistry for the synthesis of non-deuterated analogs of compounds of Formula (2) is described in Sherwood et al, J Nat Prod 2020, 83, 461-467 according to reaction Scheme (2):

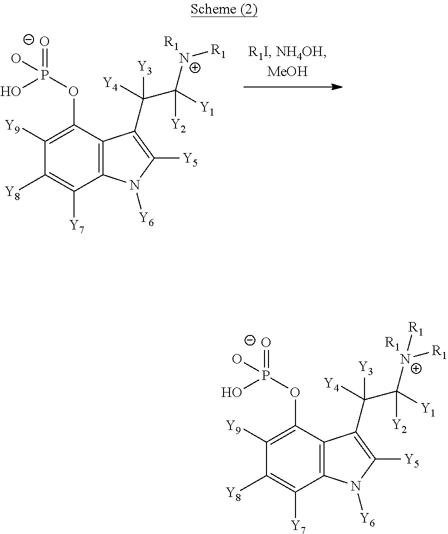

The corresponding deuterium analogs can be synthesized using suitable deuterated precursors.

In some embodiments, the present disclosure is directed to \ the compounds in Table 1:

TABLE 1
It should be noted that iodide salts are shown in the table below (compounds
1 to 22). It is recognized that other salt forms other than the iodide are
encompassed within the scope of the compounds of Table 1.

| Cpd | |
|---|---|
| Number | Structure |
| 1 | 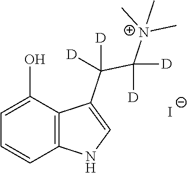 [SMILES: [2H]C([2H])(c1c[nH]c2cccc(O)c12)C([2H])([2H])[N+](C)(C)C ; [I-]] |
| 2 | 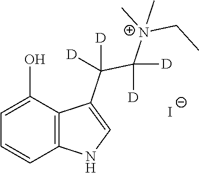 [SMILES: [2H]C([2H])(c1c[nH]c2cccc(O)c12)C([2H])([2H])[N+](C)(C)CC ; [I-]] |
| 3 | 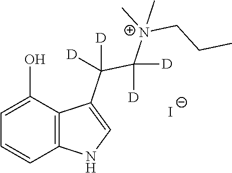 [SMILES: [2H]C([2H])(c1c[nH]c2cccc(O)c12)C([2H])([2H])[N+](C)(C)CCC ; [I-]] |
| 4 | 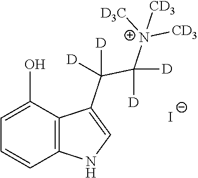 [SMILES: [2H]C([2H])(c1c[nH]c2cccc(O)c12)C([2H])([2H])[N+](C([2H])([2H])[2H])(C([2H])([2H])[2H])C([2H])([2H])[2H] ; [I-]] |
| 5 | 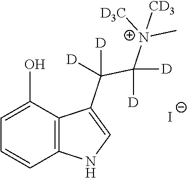 [SMILES: [2H]C([2H])(c1c[nH]c2cccc(O)c12)C([2H])([2H])[N+](C)(C([2H])([2H])[2H])C([2H])([2H])[2H] ; [I-]] |
| 6 | 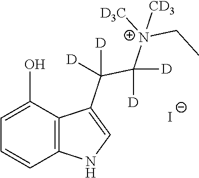 [SMILES: [2H]C([2H])(c1c[nH]c2cccc(O)c12)C([2H])([2H])[N+](CC)(C([2H])([2H])[2H])C([2H])([2H])[2H] ; [I-]] |
| 7 | 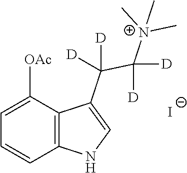 [SMILES: [2H]C([2H])(c1c[nH]c2cccc(OC(C)=O)c12)C([2H])([2H])[N+](C)(C)C ; [I-]] |
| 8 | 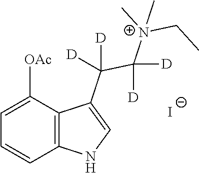 [SMILES: [2H]C([2H])(c1c[nH]c2cccc(OC(C)=O)c12)C([2H])([2H])[N+](C)(C)CC ; [I-]] |
| 9 | 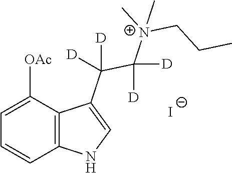 [SMILES: [2H]C([2H])(c1c[nH]c2cccc(OC(C)=O)c12)C([2H])([2H])[N+](C)(C)CCC ; [I-]] |
| 10 | 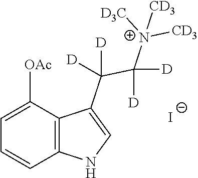 [SMILES: [2H]C([2H])(c1c[nH]c2cccc(OC(C)=O)c12)C([2H])([2H])[N+](C([2H])([2H])[2H])(C([2H])([2H])[2H])C([2H])([2H])[2H] ; [I-]] |
| 11 | 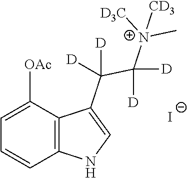 [SMILES: [2H]C([2H])(c1c[nH]c2cccc(OC(C)=O)c12)C([2H])([2H])[N+](C)(C([2H])([2H])[2H])C([2H])([2H])[2H] ; [I-]] |
| 12 | 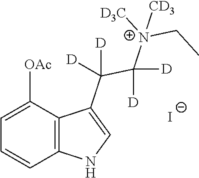 [SMILES: [2H]C([2H])(c1c[nH]c2cccc(OC(C)=O)c12)C([2H])([2H])[N+](CC)(C([2H])([2H])[2H])C([2H])([2H])[2H] ; [I-]] |
| 13 | 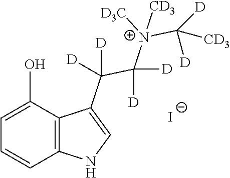 [SMILES: [2H]C([2H])([2H])C([2H])([2H])[N+](C([2H])([2H])[2H])(C([2H])([2H])[2H])C([2H])([2H])C([2H])([2H])c1c[nH]c2cccc(O)c12 ; [I-]] |
| 14 | 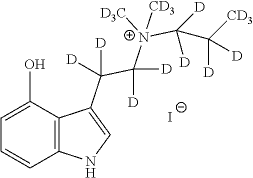 [SMILES: [2H]C([2H])([2H])C([2H])([2H])C([2H])([2H])[N+](C([2H])([2H])[2H])(C([2H])([2H])[2H])C([2H])([2H])C([2H])([2H])c1c[nH]c2cccc(O)c12 ; [I-]] |
| 15 | 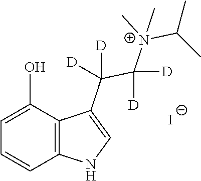 [SMILES: [2H]C([2H])(c1c[nH]c2cccc(O)c12)C([2H])([2H])[N+](C)(C)C(C)C ; [I-]] |
| 16 | 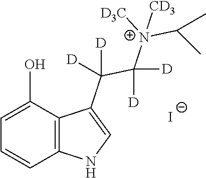 [SMILES: [2H]C([2H])(c1c[nH]c2cccc(O)c12)C([2H])([2H])[N+](C(C)C)(C([2H])([2H])[2H])C([2H])([2H])[2H] ; [I-]] |
| 17 | 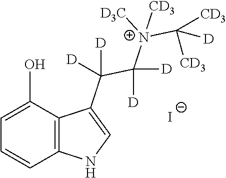 [SMILES: [2H]C([2H])([2H])C([2H])(C([2H])([2H])[2H])[N+](C([2H])([2H])[2H])(C([2H])([2H])[2H])C([2H])([2H])C([2H])([2H])c1c[nH]c2cccc(O)c12 ; [I-]] |
| 18 | 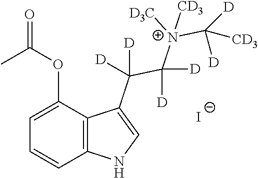 [SMILES: [2H]C([2H])([2H])C([2H])([2H])[N+](C([2H])([2H])[2H])(C([2H])([2H])[2H])C([2H])([2H])C([2H])([2H])c1c[nH]c2cccc(OC(C)=O)c12 ; [I-]] |
| 19 | 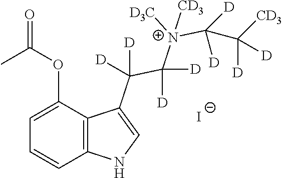 [SMILES: [2H]C([2H])([2H])C([2H])([2H])C([2H])([2H])[N+](C([2H])([2H])[2H])(C([2H])([2H])[2H])C([2H])([2H])C([2H])([2H])c1c[nH]c2cccc(OC(C)=O)c12 ; [I-]] |
| 20 | 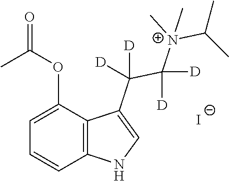 [SMILES: [2H]C([2H])(c1c[nH]c2cccc(OC(C)=O)c12)C([2H])([2H])[N+](C)(C)C(C)C ; [I-]] |
| 21 | 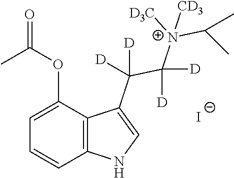 [SMILES: [2H]C([2H])(c1c[nH]c2cccc(OC(C)=O)c12)C([2H])([2H])[N+](C(C)C)(C([2H])([2H])[2H])C([2H])([2H])[2H] ; [I-]] |
| 22 | 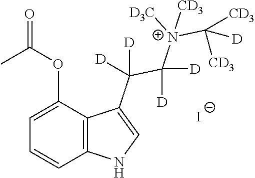 [SMILES: [2H]C([2H])([2H])C([2H])(C([2H])([2H])[2H])[N+](C([2H])([2H])[2H])(C([2H])([2H])[2H])C([2H])([2H])C([2H])([2H])c1c[nH]c2cccc(OC(C)=O)c12 ; [I-]] |
| 23 | 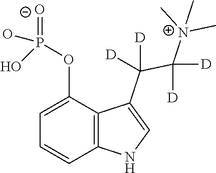 |
| 24 | 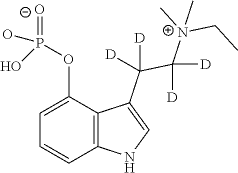 |
| 25 | 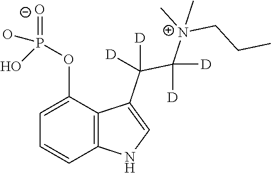 |
| 26 | 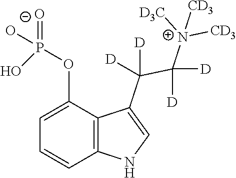 |
| 27 | 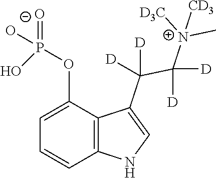 |
| 28 | 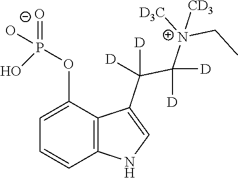 |
| 29 | 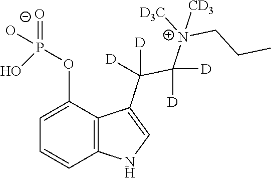 |
| 30 | 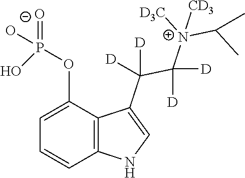 |
| 31 | 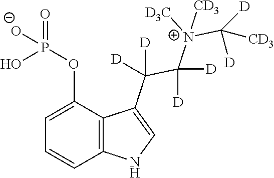 [SMILES: [2H]C([2H])([2H])C([2H])([2H])[N+](C([2H])([2H])[2H])(C([2H])([2H])[2H])C([2H])([2H])C([2H])([2H])c1c[nH]c2cccc(OP(=O)([O-])O)c12] |
| 32 | 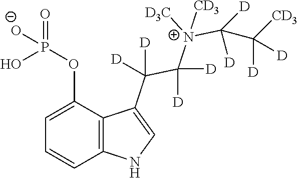 [SMILES: [2H]C([2H])([2H])C([2H])([2H])C([2H])([2H])[N+](C([2H])([2H])[2H])(C([2H])([2H])[2H])C([2H])([2H])C([2H])([2H])c1c[nH]c2cccc(OP(=O)([O-])O)c12] |
| 33 | 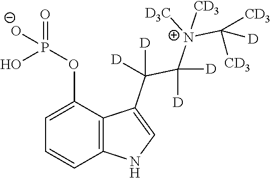 [SMILES: [2H]C([2H])([2H])C([2H])(C([2H])([2H])[2H])[N+](C([2H])([2H])[2H])(C([2H])([2H])[2H])C([2H])([2H])C([2H])([2H])c1c[nH]c2cccc(OP(=O)([O-])O)c12] |
| 34 | 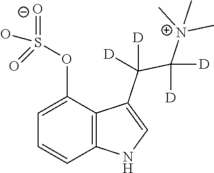 |
| 35 | 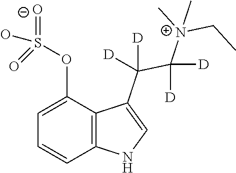 |
| 36 | 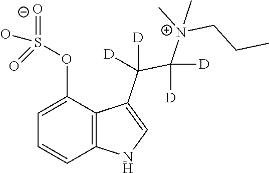 |
| 37 | 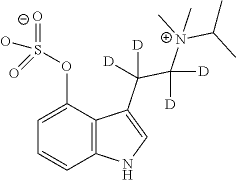 |
| 38 | 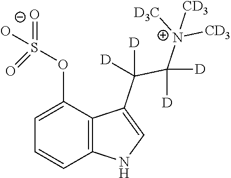 |
| 39 | 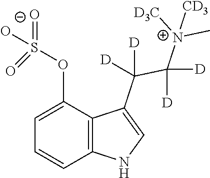 |
| 40 | 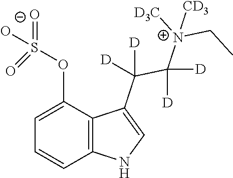 |
| 41 | 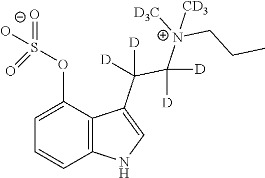 |
| 42 | 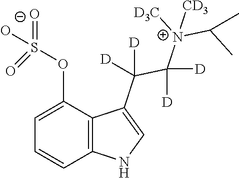 |
| 43 | 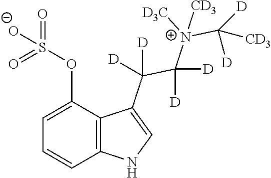 [SMILES: [2H]C([2H])([2H])C([2H])([2H])[N+](C([2H])([2H])[2H])(C([2H])([2H])[2H])C([2H])([2H])C([2H])([2H])c1c[nH]c2cccc(OS(=O)(=O)[O-])c12] |
| 44 | 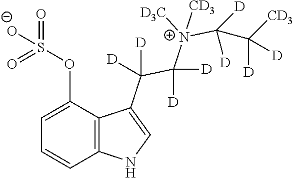 [SMILES: [2H]C([2H])([2H])C([2H])([2H])C([2H])([2H])[N+](C([2H])([2H])[2H])(C([2H])([2H])[2H])C([2H])([2H])C([2H])([2H])c1c[nH]c2cccc(OS(=O)(=O)[O-])c12] |
| 45 | 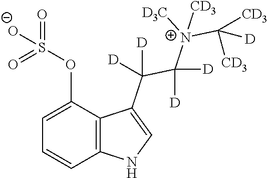 [SMILES: [2H]C([2H])([2H])C([2H])(C([2H])([2H])[2H])[N+](C([2H])([2H])[2H])(C([2H])([2H])[2H])C([2H])([2H])C([2H])([2H])c1c[nH]c2cccc(OS(=O)(=O)[O-])c12] |

Compounds provided by the present disclosure can be incorporated into pharmaceutical compositions to be administered to a patient by any appropriate route of administration including intradermal, intramuscular, intraperitoneal, intravenous, subcutaneous, intranasal, epidural, oral, peroral, sublingual, intracerebral, intravaginal, transdermal, rectal, inhalation, or topical. A pharmaceutical composition provided by the present disclosure can be an injectable formulation. A pharmaceutical composition provided by the present disclosure can be injectable intravenous formulation. A pharmaceutical composition provided by the present disclosure can be an oral formulation. An oral formulation can be an oral dosage form. A pharmaceutical composition may be formulated for intravenous administration or for subcutaneous administration.

A pharmaceutical composition provided by the present disclosure can comprise a therapeutically effective amount of a compound of Formula (2) or Table 1 together with a suitable amount of one or more pharmaceutically acceptable vehicles so as to provide a composition for proper administration to a patient. Suitable pharmaceutical vehicles and methods of preparing pharmaceutical compositions are described in the art.

A compound of Formula (2) or Table 1 and/or pharmaceutical composition thereof can generally be used in an amount effective to achieve an intended therapeutic purpose. For use to treat a disease such as a psychological disease, a compound of Formula (2) or Table 1 and/or pharmaceutical composition thereof, may be administered or applied in a therapeutically effective amount.

The amount of a compound of Formula (2) or Table 1 and/or pharmaceutical composition of any of the foregoing that will be effective in the treatment of a particular disorder or condition disclosed herein will depend in part on the nature of the disorder or condition, and can be determined by standard clinical techniques known in the art. In addition, in vitro or in vivo assays may optionally be employed to help identify optimal dosage ranges. The amount of a compound of Formula (2), and/or pharmaceutical composition of any of the foregoing administered will depend on, among other factors, the patient being treated, the weight of the patient, the severity of the affliction, the manner of administration and the judgment of the prescribing physician.

A compound of Formula (2) or Table 1 can be assayed in vitro and in vivo, for the desired therapeutic activity, prior to use in humans. For example, in vitro assays may be used to determine whether administration of a specific compound or a combination of compounds is preferred. The compounds can also be demonstrated to be effective and safe using animal model systems.

In certain embodiments, a therapeutically effective dose of a compound of Formula (2) or Table 1 and/or pharmaceutical composition of any of the foregoing will provide therapeutic benefit without causing substantial toxicity. Toxicity of a compound of Formula (2) or Table 1 and/or pharmaceutical compositions of any of the foregoing may be determined using standard pharmaceutical procedures and may be readily ascertained by the skilled artisan. The dose ratio between toxic and therapeutic effect is the therapeutic index. A compound of Formula (2) or Table 1 and/or pharmaceutical composition of any of the foregoing exhibits a particularly high therapeutic index in treating disease and disorders. A dose of a compound of Formula (2) or Table land/or pharmaceutical composition of any of the foregoing will be within a range of circulating concentrations that include an effective dose with minimal toxicity.

Compounds and pharmaceutical compositions provided by the present disclosure can be included in a kit that may be used to administer the compound to a patient for therapeutic purposes. A kit may include a pharmaceutical composition comprising a compound provided by the present disclosure suitable for administration to a patient and instructions for administering the pharmaceutical composition to the patient. The kit can be suitable for treating a psychological disease. A kit for use in treating a psychological disease, for treating a psychological disease can comprise a compound or a pharmaceutical composition provided by the present disclosure, and instructions for administering the compound to a patient.

Compounds and pharmaceutical compositions provided by the present disclosure can be included in a container, pack, or dispenser together with instructions for administration.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

Compounds provided by the present disclosure including compounds of Formula (2) and Table 1 and pharmaceutically acceptable salts thereof, and pharmaceutical compositions of any of the foregoing can be used to treat a psychological disease or disorder.

Methods of treating a psychological disease can comprise administering to a patient in need of such treatment a therapeutically effective amount of a compound of Formula (2) or Table 1 or a pharmaceutically acceptable salt thereof, or a pharmaceutical composition comprising and of the foregoing.

Examples of suitable psychological diseases include an anxiety disorder, a depressive disorder, and a compulsive disorder.

Examples of anxiety disorders include acute stress disorder, anxiety due to a medical condition, generalized anxiety disorder, panic disorder, panic attack, a phobia, post-traumatic stress disorder, separation anxiety disorder, social anxiety disorder, substance-induced anxiety disorder, and selective mutism.

Examples of depressive disorders include atypical depression, bipolar disorder, catatonic depression, depressive disorder due to a medical condition, major depressive disorder, postpartum depression, premenstrual dysphoric disorder, and seasonal affective disorder

Examples of compulsive disorders include addiction, body dysmorphic disorder, excoriation disorder, hoarding disorder, obsessive compulsive disorder, and trichotillomania.

Methods provided the present disclosure include methods of treating headaches, migraines, nicotine addiction, drug addiction, alcohol addition, compulsive disorders, psychiatric disorders, and chronic depression can comprise administering to a patient in need of such treatment a therapeutically effective amount of a compound of Formula (2) or Table 1 or a pharmaceutically acceptable salt thereof, or a pharmaceutical composition comprising and of the foregoing.

Methods of treatment provided by the present disclosure include methods of treating diseases and disorders that are capable of being treated by administering aeruginascin.

A compound provided by the present disclosure, such as a compound of Formula (2) or Table 1 can be used for increasing neuronal plasticity. A compound provided by the present disclosure can also be used to treat any brain disease. A compound provided by the present disclosure can also be used for increasing at least one of translation, transcription or secretion of neurotrophic factors.

A compound provided by the present disclosure, such as a compound of Formula (2) or Table 1, can be used to treat neurological diseases. A compound provided by the present disclosure can have, for example, anti-addictive properties, antidepressant properties, anxiolytic properties, or a combination of any of the foregoing. A neurological disease includes a neuropsychiatric disease. A neuropsychiatric disease can be a mood or anxiety disorder. Examples of neurological diseases include migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction such as substance use disorder. A neurological disease can be a migraine or cluster headache. A neurological disease can be a neurodegenerative disorder, Alzheimer's disease, or Parkinson's disease. A neurological disease can be a psychological disorder, treatment resistant depression, suicidal ideation, major depressive disorder, bipolar disorder, schizophrenia, post-traumatic stress disorder (PTSD), addiction such as substance use disorder, depression, or anxiety. A neuropsychiatric disease can be a psychological disorder, treatment resistant depression, suicidal ideation, major depressive disorder, bipolar disorder, schizophrenia, post-traumatic stress disorder (PTSD), addiction such as substance use disorder, depression, or anxiety. Examples of neuropsychiatric diseases or neurological diseases include post-traumatic stress disorder (PTSD), addiction (e.g., substance use disorder), schizophrenia, depression, and anxiety. A neuropsychiatric disease or neurological disease can be addiction such as substance use disorder. A neuropsychiatric disease or neurological disease can be depression. A neuropsychiatric disease or neurological disease can be anxiety. A neuropsychiatric disease or neurological disease can be post-traumatic stress disorder (PTSD). A neurological disease can be stroke or traumatic brain injury. A neuropsychiatric disease or neurological disease can be schizophrenia.

A compound provided by the present disclosure such as a compound of Formula (2) or Table 1 can be used for increasing neuronal plasticity. A compound provided by the present disclosure can be used to treat a brain disorder. A compound provided by the present disclosure can be used for increasing at least one of translation, transcription, or secretion of neurotrophic factors.

Method of treating a disease provided by the present disclosure include administering to a patient in need thereof, a therapeutically effective amount of a compound A compound provided by the present disclosure, such as a compound of Formula (2) or Table 1. For example, a disease can be a musculoskeletal pain disorder including fibromyalgia, muscle pain, joint stiffness, osteoarthritis, rheumatoid arthritis, or muscle cramps. Methods of treating a disease provided by the present disclosure include methods of treating women's reproductive health including premenstrual dysphoric disorder (PMDD), premenstrual syndrome (PMS), post-partum depression, and menopause.

A compound provided by the present disclosure, such as a compound of Formula (2) or Table 1, can have activity as a 5-HT_{2A} modulator A compound provided by the present disclosure can elicit a biological response by activating the 5-HT_{2A} receptor, such as allosteric modulation or modulation of a biological target that activates the 5-HT_{2A} receptor. 5-HT_{2A} agonism has been correlated with the promotion of neural plasticity. 5-HT_{2A} antagonists abrogate the neuritogenesis and spinogenesis effects of hallucinogenic compounds with 5-HT_{2A} agonist activity, for example., DMT, LSD, and DOI. A compound provided by the present disclosure can act as a 5-HT_{2A} modulator and promote neural plasticity such as cortical structural plasticity. A compound provided by the present disclosure can act as a selective 5-HT_{2A} modulator and promote neural plasticity such as cortical structural plasticity. Promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination of any of the foregoing. Increased neural plasticity can include, for example, increased cortical structural plasticity in the anterior parts of the brain.

A 5-HT_{2A} modulator such as a 5-HT_{2A} agonist can be non-hallucinogenic. A non-hallucinogenic 5-HT_{2A} modulators such as a 5-HT_{2A} agonist can be used to treat neurological diseases, which modulators do not elicit dissociative side-effects. The hallucinogenic potential of a compound provided by the present disclosure such as a compound of Formula (2) or Table 1 can be assessed in vitro. The hallucinogenic potential assessed in vitro of a compound provided by the present disclosure can be compared to the hallucinogenic potential assessed in vitro of hallucinogenic homologs. A compound provided by the present disclosure can elicit, for example, less hallucinogenic potential in vitro than the hallucinogenic homologs.

A serotonin receptor modulator, such as a modulator of serotonin receptor 2A (5-HT_{2A} modulators such as 5-HT_{2A} agonists), can be used to treat a brain disorder. A compound provided by the present disclosure such as a compound of Formula (2) or Table 1 can function as 5-HT_{2A} agonists alone, or in combination with a second therapeutic agent that also is a 5-HT_{2A} modulator. In such cases the second therapeutic agent can be an agonist or an antagonist. In some cases, it can be helpful administer a 5-HT_{2A} antagonist in combination with a compound provided by the present disclosure can be used to mitigate undesirable effects of 5-HT_{2A} agonism, such as potential hallucinogenic effects. Serotonin receptor modulators useful as second therapeutic agents for combination therapy include, for example, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, and an extended-release form of aripiprazole including Abilify Maintena, or a pharmaceutically acceptable salt, solvate, metabolite, deuterated analog, derivative, prodrug, or combinations thereof. A serotonin receptor modulator useful as a second therapeutic can be pimavanserin or a pharmaceutically acceptable salt, solvate, metabolite, derivative, or prodrug of any of the foregoing.

A serotonin receptor modulator can be administered prior to administering a compound provided by the present disclosure, such as about three or about six hours prior to administration of a compound provided by the present disclosure such as a compound of Formula (2) or Table 1. A serotonin receptor modulator can be administered prior to administering a compound provided by the present disclosure, such as about one or about three hours prior to administration of a compound provided by the present disclosure such as a compound of Formula (2) or Table 1. A serotonin receptor modulator can be administered at most about one hour prior to a compound provided by the present disclosure. In a combination therapy with a compound provided by the present disclosure, the second therapeutic agent can be a serotonin receptor modulator. A second therapeutic agent comprising a serotonin receptor modulator can be administered at a dose of from about 10 mg to about 350 mg. A serotonin receptor modulator can be provided at a dose of from about 20 mg to about 200 mg. A serotonin receptor modulator can be administered at a dose of from about 10 mg to about 100 mg. A compound provided by the present disclosure can be administered at a dose of from about 10 mg to about 100 mg, or from about 20 to about 200 mg, or from about 15 to about 300 mg, and the serotonin receptor modulator can be administered at a dose of about 10 mg to about 100 mg.

A non-hallucinogenic 5-HT_{2A} modulators (e.g., 5-HT_{2A} agonists) can be used to treat a neurological disease. Examples of neurological diseases include decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites and a combination of any of the foregoing.

A non-hallucinogenic 5-HT_{2A} modulator such as a 5-HT_{2A} agonists can be used for increasing neuronal plasticity. A non-hallucinogenic 5-HT_{2A} modulator such as 5-HT_{2A} agonists can be used for treating a brain disorder. A non-hallucinogenic 5-HT_{2A} modulator such as a 5-FIT_{2A} agonists can be used for increasing at least one of translation, transcription, or secretion of neurotrophic factors.

A compound provided by the present disclosure such as a compound of Formula (2) or Table 1 can be administered to a patient in a low dose that is lower than would produce noticeable psychedelic effects but sufficiently high to provide a therapeutic benefit. For example, the dose range can be from 200 μg to 2 mg.

A compound provided by the present disclosure such as a compound of Formula (2) or Table 1 can be used to increase neuronal plasticity. Neuronal plasticity refers to the ability of the brain to change structure and/or function throughout a subject's life. New neurons can be produced and integrated into the central nervous system throughout the subject's life. Increasing neuronal plasticity includes, for example, promoting neuronal growth, promoting neuritogenesis, promoting synaptogenesis, promoting dendritogenesis, increasing dendritic arbor complexity, increasing dendritic spine density, and increasing excitatory synapsis in the brain. Increasing neuronal plasticity can include promoting neuronal growth, promoting neuritogenesis, promoting synaptogenesis, promoting dendritogenesis, increasing dendritic arbor complexity, and increasing dendritic spine density.

Increasing neuronal plasticity by administering a compound provided by the present disclosure such as a compound of Formula (2) or Table 1 can treat, for example, neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

Methods for increasing neuronal plasticity provided by the present disclosure comprise contacting a neuronal cell with a compound provided by the present disclosure, such as a compound of Formula (2) or Table 1. Increasing neuronal plasticity can improve a brain disorder described herein.

A compound provided by the present disclosure can be used to increase neuronal plasticity. A compound provided by the present disclosure used to increase neuronal plasticity cam have, for example, anti-addictive properties, antidepressant properties, anxiolytic properties, or a combination of any of the foregoing. Decreased neuronal plasticity can be associated with a neuropsychiatric disease such as a mood or anxiety disorder. A neuropsychiatric disease includes, for example, migraine, cluster headache, post-traumatic stress disorder (PTSD), schizophrenia, anxiety, depression, and addiction such as substance abuse disorder. A brain disorder includes, for example, migraines, addiction such as substance use disorder, depression, and anxiety.

An experiment or assay used to determine increased neuronal plasticity of a compound provided by the present disclosure can be a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment such as ketanserin blocking experiments. An experiment or assay used to determine the hallucinogenic potential of a compound provided by the present disclosure can be a mouse head-twitch response (HTR) assay.

A method for increasing neuronal plasticity can comprise contacting a neuronal cell with a compound of Formula (2) or Table 1.

Methods of treating a disease include administering to a patient in need of such treatment a therapeutically effective amount of A compound provided by the present disclosure, such as a compound of Formula (2) or Table 1. The disease can be, for example, a musculoskeletal pain disorder including fibromyalgia, muscle pain, joint stiffness, osteoarthritis, rheumatoid arthritis, muscle cramps. The disease can be associated with women's reproductive health including premenstrual dysphoric disorder (PMDD), premenstrual syndrome (PMS), post-partum depression, and menopause.

Methods of treating a disease include methods of treating a brain disorder, comprising administering to a patient in need of such treatment, a therapeutically effective amount of a compound provided by the present disclosure. Methods of treating a brain disorder include a combination therapy, such as administering to a patient in need of such treatment, a therapeutically effective amount of a compound provided by the present disclosure and at least one additional therapeutic agent.

A 5-HT_{2A} modulators such as a 5-HT_{2A} agonist, can be used to treat a brain disorder. Examples of brain disorders include decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, and combinations of any of the foregoing.

A compound provided by the present disclosure such as a compound of Formula (2) or Table 1 can be used to a treat brain disorder. A compound can have, for example, anti-addictive properties, antidepressant properties, anxiolytic properties, or a combination of any of the foregoing. A brain disorder can be a neuropsychiatric disease. A neuropsychiatric disease can be a mood or anxiety disorder. Examples of brain disorders include migraine, cluster headache, post-traumatic stress disorder (PTSD), anxiety, depression, panic disorder, suicidality, schizophrenia, and addiction such as substance abuse disorder. A brain disorder can include, for example, migraine, addiction such as substance use disorder, depression, and anxiety.

Methods of treating a brain disorder can comprise administering to a patient in need of such treatment a therapeutically effective amount of a compound provided by the present disclosure such as a compound of Formula (2) or Table 1. Examples of brain disorders include a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, and substance use disorder. A brain disorder can be, for example, a neurodegenerative disorder, Alzheimer's, or Parkinson's disease. A brain disorder can be, for example, a psychological disorder, depression, addiction, anxiety, or a post-traumatic stress disorder. A brain disorder can be depression. A brain disorder can be addiction. A brain disorder can be treatment resistant depression, suicidal ideation, major depressive disorder, bipolar disorder, schizophrenia, stroke, traumatic brain injury, or substance use disorder. A brain disorder can be treatment resistant depression, suicidal ideation, major depressive disorder, bipolar disorder, schizophrenia, or substance use disorder. A brain disorder can be stroke or traumatic brain injury. A brain disorder can be treatment resistant depression, suicidal ideation, major depressive disorder, bipolar disorder, or substance use disorder. A brain disorder can be schizophrenia. A brain disorder can be alcohol use disorder.

Methods provided by the present disclosure include administering one or more additional therapeutic agents to a patient in addition to a compound of Formula (2) or Table 1 including, for example, lithium, olanzapine (Zyprexa®), quetiapine (Seroquel®), risperidone (Risperdal®), ariprazole (Abilify®), ziprasidone (Geodon®), clozapine (Clozaril®), divalproex sodium (Depakote®), lamotrigine (Lamictal®), valproic acid (Depakene®), carbamazepine (Equetro®), topiramate (Topamax®), levomilnacipran (Fetzima®), duloxetine (Cymbalta®, Yentreve®), venlafaxine (Effexor®), citalopram (Celexa®), fluvoxamine (Luvox®), escitalopram (Lexapro®), fluoxetine (Prozac®), paroxetine (Paxil®), sertraline (Zoloft®), clomipramine (Anafranil®), amitriptyline (Elavil®), desipramine (Norpramin®), imipramine (Tofranil®), nortriptyline (Pamelor®), phenelzine (Nardil®), tranylcypromine (Parnate®), diazepam (Valium®), alprazolam (Xanax®), and clonazepam (Klonopin®).

A method of treating a brain disorder can comprise administering a second therapeutic agent such as an empathogenic agent. Examples of suitable empathogenic agents for use in combination with a compound of Formula (2) or Table 1 can be selected from a phenethylamine, such as 3,4-methylene-dioxymethamphetamine (MDMA) and analogs thereof. Examples of other suitable empathogenic agents for use in combination with a compound of Formula (2) or Table 1 include:

N-allyl-3,4-methylenedioxy-amphetamine (MDAL);

N-butyl-3,4-methylenedioxy amphetamine (MDBU);

N-benzyl-3,4-methylenedioxy amphetamine (MDBZ);

N-cyclopropylmethyl-3,4-methylenedioxy amphetamine (MDCPM);

N,N-dimethyl-3,4-methylenedioxyamphetamine (MDDM);

N-ethyl-3,4-methylenedioxy amphetamine (MDE; MDEA);

N-(2-hydroxyethyl)-3,4-methylenedioxy amphetamine (MDHOET);

N-isopropyl-3,4-methylenedioxy amphetamine (MDIP);

N-methyl-3,4-ethylenedioxy amphetamine (MDMC);

N-methoxy-3,4-methylenedioxyamphetamine (MDMEO);

N-(2-methoxyethyl)-3,4-methylenedioxy amphetamine (MDMEOET);

α,α-N-trimethyl-3,4-methylenedioxyphenethylamine (MDMP;

3,4-methylenedioxy-N-methylphentermine); N-hydroxy-3,4-methylenedi oxy amphetamine (MDOH);

3,4-methylenedioxyphenethylamine (MDPEA);

α,α-dimethyl-3,4-methylenedioxyphenethylamine (MDPH; 3,4-methylenedioxyphentermine);

N-propargyl-3,4-methylenedioxy amphetamine (MDPL);

methylenedioxy-2-aminoindane (MDAI);

1,3-benzodioxolyl-N-methylbutanamine MBDB;

N-methyl-1,3-benzodioxolylbutanamine MBDB;

3,4-methylenedioxy-N-methyl-α-ethylphenylethylamine;

3,4-Methylenedioxy amphetamine MDA;

3,4-methylenedioxy-N-methylcathinone (methylone);

3,4-methylenedioxy-N-ethylcathinone (ethylone);

y hydroxy butyric acid, yhydroxybutyrate, or sodium oxybate; and

N-propyl-3,4-methylenedioxy amphetamine (MDPR).

A compound provided by the present disclosure can be used in combination with the standard of care therapy for treating a neurological disease. Examples of standard of care therapies include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, and a combination of any of the foregoing. Examples of standard of care therapies for treating depression include sertraline, fluoxetine, escitalopram, venlafaxine, and aripiprazole. Examples of standard of care therapies for treating depression include citralopram, escitalopram, fluoxetine, paroxetine, diazepam, and sertraline. Other examples of standard of care therapeutics are known to those of ordinary skill in the art.

Methods provided by the present disclosure include increasing at least one of translation, transcription, or secretion of neurotrophic factors by administering a compound of Formula (2) or Table 1 to a patient. Neurotrophic factors refers to a family of soluble peptides or proteins that support the survival, growth, and differentiation of developing and mature neurons. Increasing at least one of translation, transcription, or secretion of neurotrophic factors can be useful for increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. Increasing at least one of translation, transcription, or secretion of neurotrophic factors can increase neuronal plasticity. Increasing at least one of translation, transcription, or secretion of neurotrophic factors can promote neuronal growth, promote neuritogenesis, promote synaptogenesis, promote dendritogenesis, increase dendritic arbor complexity, and/or increase dendritic spine density.

A 5-HT_{2A} modulators such as 5-HT_{2A} agonists) can be used to increase at least one of translation, transcription, or secretion of neurotrophic factors. A compound provided by the present disclosure, such as a compound of Formula (2) or Table 1, can be used to increase at least one of translation, transcription, or secretion of neurotrophic factors. Increasing at least one of translation, transcription or secretion of neurotrophic factors can be effective in treating migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction such as substance use disorder.

An experiment or assay used to determine increased translation of neurotrophic factors includes, for example, ELISA, Western blot, immunofluorescence assays, proteomic experiments, and mass spectrometry. An experiment or assay used to determine increased transcription of neurotrophic factors includes, for example, gene expression assays, PCR, and microarrays. An experiment or assay used to determine increased secretion of neurotrophic factors includes, for example, ELISA, western blot, immunofluorescence assays, proteomic experiments, and mass spectrometry.

Methods for increasing at least one of translation, transcription or secretion of neurotrophic factors, comprise, for example, contacting a neuronal cell with a compound provided by the present disclosure, such as a compound of Formula (2) or Table 1.

Methods provided by the present disclosure include methods of treating a disease in a patient comprising administering to a patient in need thereof a therapeutically effective amount of a compound or pharmaceutical composition provided by the present disclosure, wherein the disease is a psychological disease or disorder.

Methods provided by the present disclosure include methods of treating a disease in a patient comprising administering to a patient in need thereof a therapeutically effective amount of a compound or pharmaceutical composition provided by the present disclosure, wherein the disease is a psychological disease or disorder.

The amount of a compound of Formula (2) or Table 1 provided by the present disclosure, or pharmaceutical composition thereof that will be effective in the treatment of a disease can depend, at least in part, on the nature of the disease, and may be determined by standard clinical techniques known in the art. In addition, in vitro or in vivo assays may be employed to help identify optimal dosing ranges. Dosing regimens and dosing intervals may also be determined by methods known to those skilled in the art. The amount of a compound of Formula (2) or Table 1 provided by the present disclosure administered may depend on, among other factors, the patient being treated, the weight of the patient, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of a compound of Formula (2) or Table 1 provided by the present disclosure and appropriate dosing intervals may be selected to maintain a sustained therapeutically effective concentration of a compound of Formula (2) or Table 1 provided by the present disclosure in the blood of a patient, and in certain embodiments, without exceeding a minimum adverse concentration.

A pharmaceutical composition comprising a compound of Formula (2) or Table 1 provided by the present disclosure may be administered, for example, every 4 hours, every 8 hours, every 12 hours, or once per day. Dosing may be provided alone or in combination with other drugs and may continue as long as required for effective treatment of the disease. Dosing may also be undertaken using continuous or semi-continuous administration over a period of time. Dosing includes administering a pharmaceutical composition to a mammal, such as a human, in a fed or fasted state.

A pharmaceutical composition may be administered in a single dosage form or in multiple dosage forms or as a continuous or an accumulated dose over a period of time. When multiple dosage forms are used the amount of a compound of Formula (2) or Table 1 provided by the present disclosure contained within each of the multiple dosage forms may be the same or different.

Suitable daily dosage ranges for administration can range, for example, from about 2 pg to about 200 mg of a compound of Formula (2) or Table 1 provided by the present disclosure per kilogram body weight.

Suitable daily dosage ranges for administration may range, for example, from about 1 pg to about 50 mg of a compound of Formula (2) or Table 1 provided by the present disclosure per square meter (m²) of body surface.

A compound of Formula (2) or Table 1 provided by the present disclosure may be administered to treat a psychological disease in a patient in an amount, for example, from 0.001 mg/day to 100 mg/day, or in any other appropriate daily dose. A dose can be, for example, from 0.01 μg/kg body weight/week to 100 μg/kg body weight/week or any other suitable dose.

A pharmaceutical composition comprising a compound of Formula (2) or Table 1 provided by the present disclosure may be administered to treat a psychological disease in a patient so as to provide a therapeutically effective concentration of a compound of Formula (2) or Table 1 provided by the present disclosure in the blood or plasma of the patient. A therapeutically effective concentration of a compound of a compound of Formula (2) or Table 1 provided by the present disclosure in the blood of a patient can be, for example, from 0.01 μg/L to 1,000 μg/L, from 0.1 μg/L to 500 μg/L, from 1 μg/L to 250 μg/L, or from about 10 μg/L to about 100 μg/L. A therapeutically effective concentration of a compound of Formula (2) or Table 1 provided by the present disclosure in the blood of a patient can be, for example, at least 0.01 μg/L, at least 0.1 μg/L, at least 1 μg/L, at least about 10 μg/L, or at least 100 μg/L. A therapeutically effective concentration of a compound of Formula (2) or Table 1 in the blood of a patient can be, for example, less than an amount that causes unacceptable adverse effects including adverse effects to homeostasis. A therapeutically effective concentration of a compound of Formula (2) or Table 1 in the blood of a patient can be an amount sufficient to restore and/or maintain homeostasis in the patient.

Pharmaceutical compositions provided by the present disclosure may be administered to treat a disease in a patient so as to provide a therapeutically effective concentration of a compound of Formula (2) or Table 1 in the blood of a patient for a period of time such as, for example, for 4 hours, 8 hours, 12 hours, 16 hours, 20 hours, 1 day, or 2 days.

The amount of a compound of Formula (2) or Table 1 administered may vary during a treatment regimen.

Pharmaceutical compositions provided by the present disclosure may further comprise one or more pharmaceutically active compounds in addition to a compound of Formula (2) or Table 1. Such compounds may be provided, for example, to treat the psychological disease being treated with the compound of Formula (2) or Table 1 or to treat a disease, disorder, or condition other than the psychological disease being treated with the compound of Formula (2) or Table 1, to treat a side-effect caused by administering the compound of Formula (2) or Table 1, to augment the efficacy of the compound of Formula (2) or Table 1, and/or to modulate the activity of the compound of Formula (2) or Table 1.

A compound of Formula (2) or Table 1 provided by the present disclosure may be administered in combination with at least one other therapeutic agent. A compound of Formula (2) or Table 1 may be administered to a patient together with another compound for treating a psychological disease in the patient. The at least one other therapeutic agent can be a second, different compound of Formula (2) or Table 1. A compound of Formula (2) or Table 1 and the at least one other therapeutic agent may act additively or, and in certain embodiments, synergistically with another compound of Formula (2) or Table 1. The at least one additional therapeutic agent may be included in the same pharmaceutical composition or vehicle comprising the compound of Formula (2) or Table 1 or may be in a separate pharmaceutical composition or vehicle. Accordingly, methods provided by the present disclosure further include, in addition to administering a compound of Formula (2) or Table 1, administering one or more therapeutic agents effective for treating a psychological disease or a different disease, disorder or condition than a psychological disease. Methods provided by the present disclosure include administration of a compound of Formula (2) or Table 1 and one or more other therapeutic agents provided that the combined administration does not inhibit the therapeutic efficacy of the compound of Formula (2) or Table 1 and/or does not produce adverse combination effects.

A pharmaceutical composition comprising a compound of Formula (2) or Table 1 may be administered concurrently with the administration of another therapeutic agent, which may be part of the same pharmaceutical composition as, or in a different pharmaceutical composition than that comprising a compound of Formula (2) or Table 1. A compound of Formula (2) or Table 1 may be administered prior or subsequent to administration of another therapeutic agent. In certain combination therapies, the combination therapy may comprise alternating between administering a compound of Formula (2) or Table 1 and a composition comprising another therapeutic agent, e.g., to minimize adverse drug effects associated with a particular drug. When a compound of Formula (2) or Table 1 is administered concurrently with another therapeutic agent that potentially may produce an adverse drug effect including, for example, toxicity, the other therapeutic agent may be administered at a dose that falls below the threshold at which the adverse drug reaction is elicited.

A pharmaceutical composition comprising a compound of Formula (2) or Table 1 provided by the present disclosure may be administered with one or more substances, for example, to enhance, modulate and/or control release, bioavailability, therapeutic efficacy, therapeutic potency, and/or stability, of the compound of Formula (2) or Table 1. For example, a pharmaceutical composition comprising a compound of Formula (2) or Table 1 can be co-administered with an active agent having pharmacological effects that enhance the therapeutic efficacy of the compound of Formula (2) or Table 1.

For example, a compound of Formula (2) or Table 1 can be co-administered together with a therapeutically effective amount of a psilocybin derivative, a cannabinoid, a serotonergic drug, an adrenergic drug, a dopaminergic drug, an anxiolytic drug, an antidepressant or a combination of any of the foregoing to treat a psychological disease in a patient.

Examples of suitable psilocybin derivatives include [3-(2-dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Examples of suitable cannabinoids include cannabichromene (CBC), cannabichromenic acid (CBCA), cannabichromevarin (CBCV), cannabichromevarinic acid (CB-CVA), cannabicyclol (CBL), cannabicyclolic acid (CBLA), cannabicyclovarin (CBLV), cannabidiol (CBD), cannabidiol monomethylether (CBDM), cannabidiolic acid (CBDA), cannabidiorcol (CBD-Cl), cannabidivarin (CBDV), cannabidivarinic acid (CBDVA), cannabielsoic acid B (CBEA-B), cannabielsoin (CBE), cannabielsoin acid A (CBEA-A), cannabigerol (CBG), cannabigerol monomethylether (CBGM), cannabigerolic acid (CBGA), cannabigerolic acid monomethylether (CBGAM), cannab-igerovarin (CBGV), cannabigerovarinic acid (CBGVA), cannabinodiol (CBND), cannabinodivarin (CBDV), can nabinol (CBN), cannabinol methylether (CBNM), cannabinol-C2 (CBN-C2), cannabinol-C4 (CBN-C4), cannabinolic acid (CBNA), cannabiorcool (CBN-Cl), cannabivarin (CBV), cannabitriol (CBT), cannabitriolvarin (CBTV), 10-ethoxy-9-hydroxy-ö-6a-tetrahydrocannabinol, cannbicitran (CBT), cannabiripsol (CBR), 8,9-dihydroxy-ö-6a-tetrahydrocannabinol, ö-8-tetrahydrocannabinol (118-THC), ö-8-tetrahydrocannabinolic acid (118-THCA), ö-9-tetrahydrocannabinol (THC), ö-9-tetrahydrocannabinol-C4 (THC-C4), ö-9-tetrahydrocannabinolic acid A (THCA-A), ö-9-tetrahydrocannabinolic acid B (THCA-B), ö-9-tetrahydrocannabinolic acid-C4 (THCA-C4), ö-9-tetrahydrocannabiorcol (THC-Cl), ö-9-tetrahydrocannabiorcolic acid (THCA-Cl), ö-9-tetrahydrocannabivarin (THCV), 6 tetrahydrocannabivarinic acid (THCVA), 10-oxo-ö-6a-tetrahydrocannabinol (OTHC), cannabichromanon (CBCF), cannabifuran (CBF), cannabiglendol, ö-9 cis-tetrahydrocannabinol (cis-THC), tryhydroxy-ö-9-tetra-hydrocannabinol (triOH-THC), dehydrocannabifuran (DCBF), and 3,4,5,6-tetrahydro-7-hydroxy-α-α-2-trimethyl-9-n-propyl-2,6-methano-2H-1-benzoxocin-5-methanol.

Examples of suitable serotonergic drugs include 6-allyl-N,N-diethylnl, N,N-dibutyl-tryptamine, N,N-diethyl-tryptamine, N,N-diisopropyl-tryptamine, 5-methyoxy-α-methyl-tryptamine, N,N-dimethyl-tryptamine, 2,α-dimethyl-tryptamine, α,n-dimethyl-tryptamine, N,N-dipropyl-tryptamine, n-ethyl-n-isopropyl-tryptamine, α-ethyl-tryptamine, 6, N,N-triethylnl, 3,4-dihydro-7-methoxy-1-methyl-c, 7-methyloxy-1-methyl-c, N,N-dibutyl-4-hydroxy-tryptamine, N,N-diethyl-4-hydroxy-tryptamine, N,N-diisopropyl-4-hydroxy-tryptamine, N,N-dimethyl-4-hydroxy-tryptamine, N,N-dimethyl-5-hydroxy-tryptamine, N,N-dipropyl-4-hydroxy-tryptamine, n-ethyl-4-hydroxy-n-methyl-tryptamine, 4-hydroxy-n-isopropyl-n-methyl-tryptamine, 4-hydroxy-n-methyl-n-propyl-tryptamine, 4-hydroxy-N,N-tetramethylene-tryptamine ibogaine, N,N-diethyl-1, n-butyl-n-methyl-tryptamine, N,N-diisopropyl-4,5-methylenedioxy-tryptamine, N,N-diisopropyl-5,6-methylenedioxy-tryptamine, N,N-dimethyl-4,5-methylenedioxy-tryptamine, N,N-dimethyl-5,6-methylenedioxy-tryptamine, n-isopropyl-n-methyl-5,6-methylenedioxy-tryptamine, N,N-diethyl-2-methyl-tryptamine, 2, N,N-trimethyl-tryptamine, n-acetyl-5-methoxy-tryptamine, N,N-diethyl-5-methoxy-tryptamine, N,N-diisopropyl-5-methoxy-tryptamine, 5-methoxy-n, n-dimethyl-tryptamine, n-isopropyl-4-methoxy-n-methyl-tryptamine, n-iso-propyl-5-methoxy-n-methyl-tryptamine, 5,6-dimethoxy-n-isopropyl-n-methyl-tryptamine, 5-methoxy-n-methyl-tryptamine, 5-methoxy-N,N-tetramethylene-tryptamine, 6-methoxy-1-methyl-1,2,3,4-tetrahydro-c, 5-methoxy-2, N,N-trimethyl-tryptamine, N,N-dimethyl-5-methylthio-tryptamine, n-isopropyl-n-methyl-tryptamine, α-methyl-tryptamine, n-ethyl-tryptamine, n-methyl-tryptamine, 6-propylnl, N,N-tetramethylene-tryptamine, tryptamine, and 7-methoxy-1-methyl-1,2,3,4-tetrahydro-c, and n-dimethyl-5-methoxy-tryptamine.

Examples of suitable serotonergic drugs further include alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, and vortioxetine.

Examples of suitable adrenergic drugs include adrenaline, agmatine, amoxapine, aptazapine, atomoxetine, bupropion, clonidine, doxepin, duloxetine, esmirtazpine, mianserin, mirabegron, mirtazapine, norepinephrine, phentolamine, phenylephrine, piperoxan, reserpine, ritodrine, setiptiline, tesofensine, timolol, trazodone, trimipramine, and xylazine.

Examples of suitable dopaminergic drugs include amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psycho-stimulant, reserpine, risperidone, ropinirole, tetrabenazine, and thioridazine.

Examples of suitable anxiolytic drugs include alprazolam, an a blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, and triazolam.

Examples of suitable antidepressants include bupropion, citalopram, duloxetine, escitalopram, fluoxetine, fluvoxamine, milnacipran, mirtazapine, paroxetine, reboxetine, sertraline, and venlafaxine.

It should be understood that any suitable combination of the compounds and pharmaceutical compositions provided herein with one or more of the above therapeutic agents and optionally one or more further pharmacologically active substances are considered to be within the scope of the present disclosure. In some embodiments, the compounds and pharmaceutical compositions provided by the present disclosure can be administered prior to or subsequent to the one or more additional active ingredients.

### Aspects of the Present Disclosure

Aspect 1. A compound of Formula (2),

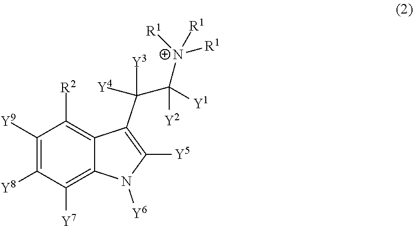

or a pharmaceutically acceptable salt thereof, wherein,

each IV is independently selected from CH₃, CH₂D, CHD₂, and CD₃;

R² is selected from an oxygen radical, a phosphate radical, a sulfate radical, and the moiety —O—C(═O)—R³ wherein R³ is selected from CH₃, CH₂D, CHD₂, and CD₃;

each of Y¹, Y², Y³, Y⁴, Y⁵, Y⁶, Y⁷, Y⁸, and Y⁹ is independently selected from hydrogen and deuterium; and

one or more of R¹, Y¹, Y², Y³, Y⁴, Y⁵, Y⁶, Y⁷, Y⁸, and Y⁹ comprises deuterium.

Aspect 2. The compound of aspect 1, wherein the compound has the structure of Formula (2a), Formula (2b), Formula (2c), or Formula (2d):

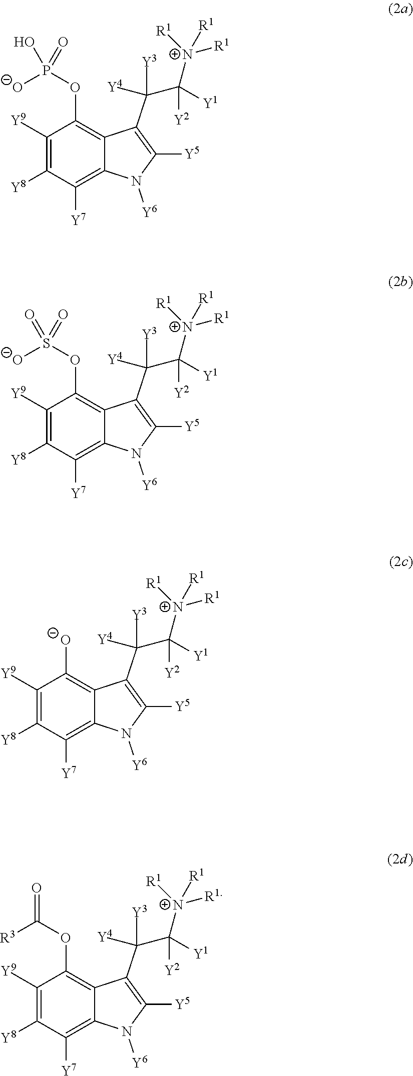

Aspect 3. The compound of aspect 2, wherein the compound has the structure of Formula (2a).

Aspect 4. The compound of aspect 2, wherein the compound has the structure of Formula (2b).

Aspect 5. The compound of aspect 2, wherein the compound has the structure of Formula (2c).

Aspect 6. The compound of aspect 2, wherein the compound has the structure of Formula (2d).

Aspect 7. The compound of any one of aspects 1 to 6, wherein R² is an oxygen radical (—O).

Aspect 8. The compound of any one of aspects 1 to 6, wherein R² is a phosphate radical (—OP(═O)(—OH)(—O⁻)

Aspect 9. The compound of any one of aspects 1 to 6, wherein R² is a sulfate radical (—OS(═O)₂(—O⁻)

Aspect 10. The compound of any one of aspects 1 to 6, wherein R² is —O—C(═O)—R³.

Aspect 11. The compound of any one of aspects 1 to 6, wherein R² is —O—C(═O)—R³, and R³ is CH₃.

Aspect 12. The compound of any one of aspects 1 to 6, wherein R² is —O—C(═O)—R³, and R³ is CH₂D.

Aspect 13. The compound of any one of aspects 1 to 6, wherein R² is —O—C(═O)—R³, and R³ is CHD₂.

Aspect 14. The compound of any one of aspects 1 to 6, wherein R² is —O—C(═O)—R³, and R³ is CD₃.

Aspect 15. The compound of any one of aspects 1 to 14, wherein each of Y⁵, Y⁶, Y⁷, Y⁸, and Y⁹ is hydrogen.

Aspect 16. The compound of any one of aspects 1 to 14, wherein each of Y⁵, Y⁶, Y⁷, Y⁸, and Y⁹ is deuterium.

Aspect 17. The compound of any one of aspects 1 to 14, wherein one of Y⁵, Y⁶, Y⁷, Y⁸, and Y⁹ is hydrogen and each of the other of Y⁵, Y⁶, Y⁷, Y⁸, and Y⁹ is deuterium.

Aspect 18. The compound of any one of aspects 1 to 14, wherein two of Y⁵, Y⁶, Y⁷, Y⁸, and Y⁹ is hydrogen and each of the other of Y⁵, Y⁶, Y⁷, Y⁸, and Y⁹ is deuterium.

Aspect 19. The compound of any one of aspects 1 to 14, wherein three of Y⁵, Y⁶, Y⁷, Y⁸, and Y⁹ is hydrogen and the other of Y⁵, Y⁶, Y⁷, Y⁸, and Y⁹ is deuterium.

Aspect 20. The compound of any one of aspects 1 to 14, wherein four of Y⁵, Y⁶, Y⁷, Y⁸, and Y⁹ is hydrogen and the other of Y⁵, Y⁶, Y⁷, Y⁸, and Y⁹ is deuterium.

Aspect 21. The compound of any one of aspects 1 to 20, wherein each R¹ is independently selected from CH₃, CH₂D, CHD₂, and CD₃.

Aspect 22. The compound of any one of aspects 1 to 20, wherein each R¹ is independently selected from CH₃ and CD₃.

Aspect 23. The compound of any one of aspects 1 to 20, wherein each R¹ is CH₃.

Aspect 24. The compound of any one of aspects 1 to 20, wherein each R¹ is CD₃.

Aspect 25. The compound of any one of aspects 1 to 24, wherein each of Y¹, Y², Y³, and Y⁴ is deuterium.

Aspect 26. The compound of any one of aspects 1 to 24, wherein each of Y¹ and Y² is deuterium, and each of Y³ and Y⁴ is hydrogen.

Aspect 27. The compound of any one of aspects 1 to 24, wherein each of Y¹ and Y² is hydrogen, and each of Y³ and Y⁴ is deuterium.

Aspect 28. The compound of any one of aspects 1 to 24, wherein each of Y¹, Y², and Y³ is hydrogen, and Y⁴ is deuterium.

Aspect 29. The compound of any one of aspects 1 to 24, wherein each of Y² and Y³ is hydrogen, and each of Y¹ and Y⁴ is deuterium.

Aspect 30. The compound of any one of aspects 1 to 24, wherein Y² is hydrogen, and each of Y¹, Y³ and Y⁴ is deuterium.

Aspect 31. The compound of any one of aspects 1 to 24, wherein Y³ is hydrogen, and each of Y¹, Y² and Y⁴ is deuterium.

Aspect 32. The compound of any one of aspects 1 to 24, wherein Y¹, Y², Y³, and Y⁴ is hydrogen.

Aspect 33. The compound of any one of aspects 1 to 32, wherein the compound is not selected from:

3-(2-(bis(methyl-d3)amino)ethyl-1,1,2,2-d4)-1H-indol-4-ol (3a);

3-(2-(dimethylamino)ethyl-1,1,2,2-d4)-1H-indol-4-ol (3b);

3-(2-(bis(methyl-d3)amino)ethyl)-1H-indol-4-ol (3c);

3-(2-(dimethylamino)ethyl-1,1,2,2-d4)-1H-indol-4-yl acetate (3d);

3-(2-(bis(methyl-d3)amino)ethyl-1,1,2,2-d4)-1H-indol-4-yl acetate (3e);

3-(2-(bis(methyl-d3)amino)ethyl)-1H-indol-4-yl acetate (30;

3-(2-(dimethylamino)ethyl-1,1,2,2-d4)-1H-indol-4-yl hydrogen phosphate (3g);

3-(2-(bis(methyl-d3)amino)ethyl-1,1,2,2-d4)-1H-indol-4-yl hydrogen phosphate (3h); and

3-(2-(bis(methyl-d3)amino)ethyl)-1H-indol-4-yl hydrogen phosphate (3i).

Aspect 34. A pharmaceutical composition comprising the compound of any one of aspects 1 to 33, or a pharmaceutically acceptable salt thereof.

Aspect 35. The pharmaceutical composition of aspect 34, wherein the pharmaceutical composition is an oral pharmaceutical composition.

Aspect 36. The pharmaceutical composition of any one of aspects 34 to 35, wherein the pharmaceutical composition comprises a serotonergic drug, a psilocybin derivative, a cannabinoid, or a terpene.

Aspect 37. A method treating a disease in a patient comprising administering to a patient in need of such treatment a therapeutically effective amount of the compound of any one of aspects 1 to 33, or a pharmaceutically acceptable salt thereof or the pharmaceutical composition of any one of aspects 34 to 35, wherein the disease is selected from a psychological disease, an inflammatory disease, pain, a brain disease, and a developmental disease.

Aspect 38. The method of aspect 37, wherein the method comprises co-administering a therapeutically effective amount of a compound selected from a psilocybin derivative, a cannabinoid, a serotonergic drug, a dopaminergic drug, an anti-depressant, an anxiolytic drug, or a combination of any of the foregoing.

Aspect 39. The method of any one of aspects 36 to 37, wherein administering comprises orally administering.

Aspect 40. Use of the compound of any one of aspects 1 to 33, or the pharmaceutically acceptable salt thereof or a pharmaceutical composition of any one of aspects 34 to 35, in the manufacture of a medicament for treating a disease is a psychological disease.

### EXAMPLES

The following examples describe in detail the synthesis of compounds of Formula (2), the characterization of compounds of Formula (2), and uses of compounds of Formula (2). It will be apparent to those skilled in the art that many modifications, both to materials and methods, may be practiced without departing from the scope of the disclosure.

### Example 1

Methods of Synthesizing Compounds of Formula (2)

Compounds of Formula (2) can be synthesized using the methods described in Chadeayne et al, ACS Omega 2020, 5, 16940-16943 and Sherwood et al, J Nat Prod 2020, 83, 461-467 using suitable deuterated precursors.

Synthesis of 3-(2-(Dimethylamino)ethyl-1,1,2,2-d₄)-1H-indol-4-yl acetate

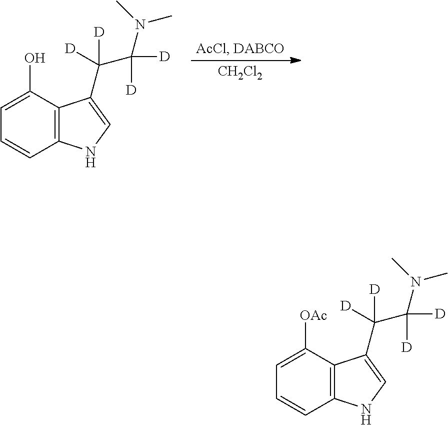

To a suspension of 3-(2-(dimethylamino)ethyl-1,1,2,2-d₄)-1H-indol-4-ol [CAS No: 1286546-49-3] (430 mg, 2.06 mmol, 1.0 equiv.) and 1,4-diazabicyclo[2.2.2]octane (24 mg, 0.21 mmol, 0.1 equiv.) in anhydrous dichloromethane (2 mL) was added dropwise, acetyl chloride (275 mg, 250 μL, 3.51 mmol, 1.7 equiv.) at 0° C. under nitrogen. The mixture was stirred at 0° C. for 45 min and then at room temperature for 2 h. The reaction mixture was quenched with saturated aqueous NaHCO₃(9 mL) and extracted with ethyl acetate (3×20 mL) and dichloromethane (3×20 mL). The combined organic layers were dried (MgSO₄) and concentrated to give 3-(2-(dimethylamino)ethyl-1,1,2,2-4-1H-indol-4-yl acetate (505 mg, 98%) as an oil. LCMS (+ve mode): m/z=251.15 [M+H]⁺; ¹H NMR (CDCl₃, 300 MHz) δ 8.22 (s, 1H, NH), 7.15 (m, 2H, 2×ArH), 6.94 (d, 1H, J=2.4 Hz, ArH), 6.80 (dd, 1H, J=7.4, 0.9 Hz, ArH), 2.40 (s, 3H, CH₃), 2.32 (s, 6H, 2×CH₃).Synthesis of 4-Acetoxy-3-(2-(trimethyl-λ⁴-azaneyl)ethyl-1,1,2,2-d₄)-1H-indole iodide (compound 7)

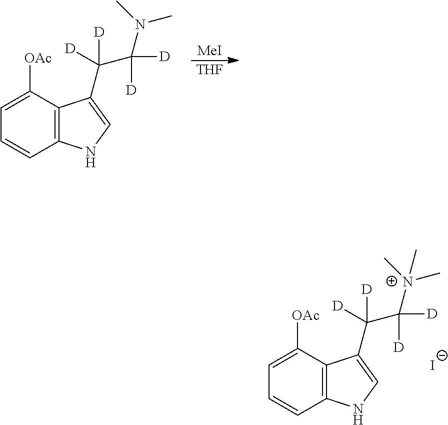 [SMILES: "MeI" ; [2H]C([2H])(c1c[nH]c2cccc(OC(C)=O)c12)C([2H])([2H])N(C)C ; [2H]C([2H])(c1c[nH]c2cccc(OC(C)=O)c12)C([2H])([2H])[N+](C)(C)C ; [I-] ; "THF"]

To a solution of 3-(2-(dimethylamino)ethyl-1,1,2,2-c/4)-1H-indol-4-yl acetate (130 mg, 0.52 mmol, 1.0 equiv.) in anhydrous tetrahydrofuran (15 mL) was added methyl iodide (6.80 g, 3.0 mL, 48.2 mmol, 92.9 equiv.). The mixture was heated at 66° C. for 18 h under nitrogen in the dark in a sealed microwave vessel. The resulting suspension was cooled to room temperature. The precipitate was isolated via vacuum filtration and washed with diethyl ether (4×2 mL) to afford 4-acetoxy-3-(2-(trimethyl-λ⁴-azaneyl)ethyl-1,1,2,2-d₄)-1H-indole iodide (165 mg, 81%) as a solid. LCMS (+ve mode): m/z=265.20 [M+H]⁺; ¹H NMR (D20, 300 MHz) δ 7.46 (m, 1H, ArH), 7.31 (s, 1H, ArH), 7.25 (t, 1H, J=8.0 Hz, ArH), 6.89 (d, 1H, J=7.7 Hz, ArH), 3.19 (s, 9H, 3×CH₃), 2.46 (s, 3H, CH₃).Synthesis of 4-Hydroxy-3-(2-(trimethyl-λ⁴-azaneyl)ethyl-1,1,2,2-d₄)-1H-indole iodide (compound 1)

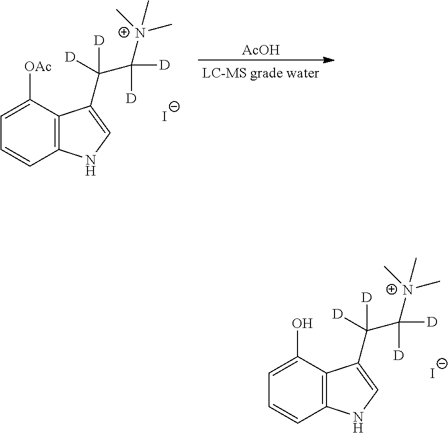 [SMILES: "AcOH" ; [2H]C([2H])(c1c[nH]c2cccc(OC(C)=O)c12)C([2H])([2H])[N+](C)(C)C ; [I-] ; [2H]C([2H])(c1c[nH]c2cccc(O)c12)C([2H])([2H])[N+](C)(C)C ; [I-]]

To a suspension of 4-acetoxy-3-(2-(trimethyl-λ⁴-azaneyl)ethyl-1,1,2,2-d₄)-1H-indole iodide (160 mg, 0.41 mmol, 1.0 equiv.) in LC-MS grade water (1.5 mL) was added acetic acid (5.30 g, 5.0 mL, 87.3 mmol, 214.0 equiv.). The mixture was heated at 100° C. for 18 h under nitrogen in the dark in a sealed microwave vessel. The resulting solution was cooled to room temperature and concentrated. The resulting brown semi-solid was sonicated in a small volume (4 mL) of a 1:1 mixture of tetrahydrofuran and acetone. Petrol (10 mL) was added to the solution, producing a precipitate. The solid was isolated via vacuum filtration and washed with petrol (3×3 mL) to afford 4-hydroxy-3-(2-(trimethyl-λ⁴-azaneyl)ethyl-1,1,2,2-d₄)-1H-indole iodide (100 mg, 70%) as a solid. LCMS (+ve mode): m/z=223.15 [M+H]⁺; ¹H NMR (D20, 300 MHz) δ 7.17 (s, 1H, ArH), 7.09 (m, 2H, 2×ArH), 6.56 (dd, 1H, J=5.5, 2.9 Hz, ArH), 3.19 (s, 9H, 3×CH₃).

Synthesis of 4-Acetoxy-3-(2-(ethyldimethyl-λ⁴-azaneyl)ethyl-1,1,2,2-d₄)-1H-indole iodide (compound 8)

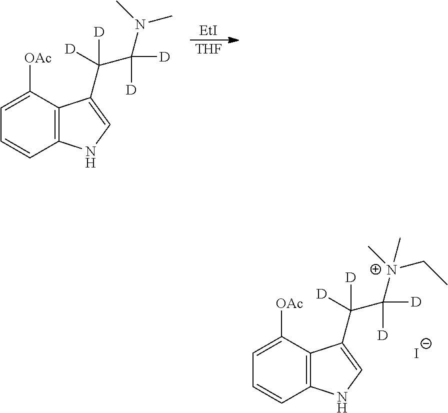 [SMILES: "EtI" ; [2H]C([2H])(c1c[nH]c2cccc(OC(C)=O)c12)C([2H])([2H])N(C)C ; [2H]C([2H])(c1c[nH]c2cccc(OC(C)=O)c12)C([2H])([2H])[N+](C)(C)CC ; [I-] ; "THF"]

To a solution of 3-(2-(dimethylamino)ethyl-1,1,2,2-d₄)-1H-indol-4-yl acetate (130 mg, 0.52 mmol, 1.0 equiv.) in anhydrous tetrahydrofuran (15 mL) was added ethyl iodide (5.80 g, 3.0 mL, 37.3 mmol, 80 equiv.). The mixture was heated at 66° C. for 18 h under nitrogen in the dark in a sealed microwave vessel. The resulting suspension was cooled to room temperature. The precipitate was isolated via vacuum filtration and washed with diethyl ether (3×3 mL) to afford 4-acetoxy-3-(2-(ethyldimethyl-λ⁴-azaneyl)ethyl-1,1,2,2-d₄)-1H-indole iodide (150 mg, 74%) as a solid. LCMS (+ve mode): m/z=279.20 [M+H]⁺; ¹H NMR (D20, 300 MHz) δ 7.46 (d, 1H, J=8.2 Hz, ArH), 7.31 (s, 1H, ArH), 7.25 (t, 1H, J=8.0 Hz, ArH), 6.89 (d, 1H, J=7.7 Hz, ArH), 3.44 (q, 2H, J=7.3 Hz, CH₂), 3.10 (s, 6H, 2×CH₃), 2.46 (s, 3H, CH₃), 1.35 (t, 3H, J=7.3 Hz, CH₃).

Synthesis of 3-(2-(Ethyldimethyl-λ⁴-azaneyl)ethyl-1,1,2,2-d₄)-4-hydroxy-1H-indole iodide (compound 2)

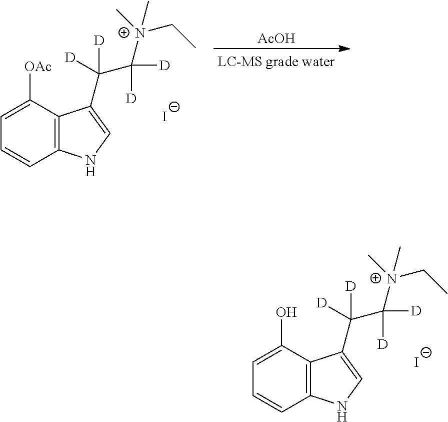 [SMILES: "AcOH" ; [2H]C([2H])(c1c[nH]c2cccc(OC(C)=O)c12)C([2H])([2H])[N+](C)(C)CC ; [I-] ; [2H]C([2H])(c1c[nH]c2cccc(O)c12)C([2H])([2H])[N+](C)(C)CC ; [I-]]

To a suspension of 4-acetoxy-3-(2-(ethyldimethyl-λ⁴-azaneyl)ethyl-1,1,2,2-d₄)-1H-indole iodide (145 mg, 0.36 mmol, 1.0 equiv.) in LC-MS grade water (1.5 mL) was added acetic acid (5.30 g, 5.0 mL, 87.3 mmol, 244.5 equiv.). The mixture was heated at 100° C. for 18 h under nitrogen in the dark in a sealed microwave vessel. The resulting solution was cooled to room temperature and concentrated. The resulting brown semi-solid was sonicated in a small volume (5 mL) of a 1:1 mixture of tetrahydrofuran and acetone. Petrol (10 mL) was added to the solution, producing a precipitate. The solid was isolated via vacuum filtration and washed with petrol (3×5 mL) to afford 3-(2-(ethyldimethyl-λ⁴-azaneyl)ethyl-1,1,2,2-d₄)-4-hydroxy-1H-indole iodide (55 mg, 42%) as a solid. LCMS (+ve mode): m/z=237.15 [M+H]⁺; ¹H NMR (D20, 300 MHz) δ 7.17 (s, 1H, ArH), 7.09 (m, 2H, 2×ArH), 6.55 (dd, 1H, J=5.5, 2.9 Hz, ArH), 3.43 (q, 2H, J=7.3 Hz, CH₂), 3.10 (s, 6H, 3×CH₃), 1.37 (t, 3H, J=7.3 Hz, CH₃).

Synthesis of 4-Acetoxy-3-(2-(dimethyl(propyl)-λ⁴-azaneyl)ethyl-1,1,2,2-d₄)-1H-indole iodide (compound 9)

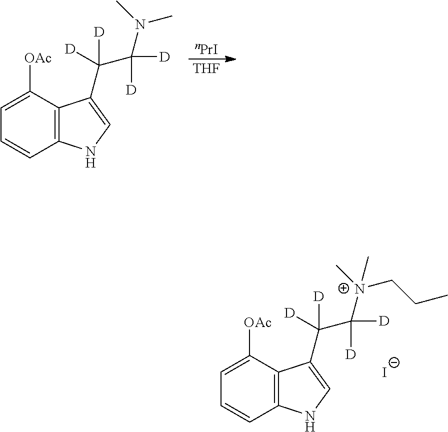

To a solution of 3-(2-(dimethylamino)ethyl-1,1,2,2-d₄)-1H-indol-4-yl acetate (130 mg, 0.52 mmol, 1.0 equiv.) in anhydrous tetrahydrofuran (15 mL) was added n-propyl iodide (5.3 g, 3.0 mL, 30.9 mmol, 59.5 equiv.). The mixture was heated at 66° C. for 18 h under nitrogen in the dark in a sealed microwave vessel. The resulting suspension was cooled to room temperature. The precipitate was isolated via vacuum filtration and washed with diethyl ether (3×3 mL) to afford 4-acetoxy-3-(2-(dimethyl(propyl)-λ⁴-azaneyl)ethyl-1,1,2,2-d₄)-1H-indole iodide (132 mg, 61%) as a solid. LCMS (+ve mode): m/z=293.15 [M+H]⁺; ¹H NMR (D20, 300 MHz) δ 7.46 (d, 1H, J=8.2 Hz, ArH), 7.32 (s, 1H, ArH), 7.25 (t, 1H, J=8.0 Hz, ArH), 6.89 (d, 1H, J=7.7 Hz, ArH), 3.29 (m, 2H, CH₂), 3.12 (s, 6H, 2×CH₃), 2.47 (s, 3H, CH₃), 1.76 (m, 2H, CH₂), 0.91 (t, 3H, J=7.3 Hz, CH₃).

Synthesis of 3-(2-(Dimethyl(propyl)-λ⁴-azaneyl)ethyl-1,1,2,2-d₄)-4-hydroxy-1H-indole iodide (compound 3)

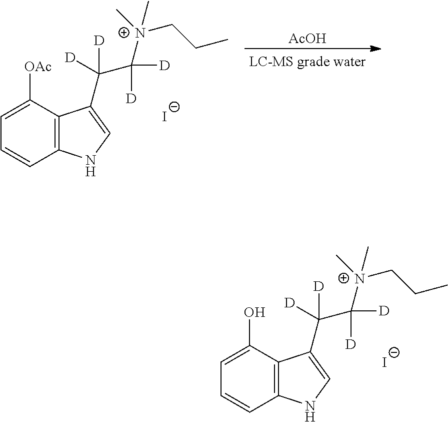 [SMILES: "AcOH" ; [2H]C([2H])(c1c[nH]c2cccc(OC(C)=O)c12)C([2H])([2H])[N+](C)(C)CCC ; [I-] ; [2H]C([2H])(c1c[nH]c2cccc(O)c12)C([2H])([2H])[N+](C)(C)CCC ; [I-]]

To a suspension of 4-acetoxy-3-(2-(dimethyl(propyl)-λ⁴-azaneyl)ethyl-1,1,2,2-d₄)-1H-indole iodide (132 mg, 0.31 mmol, 1.0 equiv.) in LC-MS grade water (1.5 mL) was added acetic acid (5.3 g, 5.0 mL, 87.3 mmol, 278.0 equiv.). The mixture was heated at 100° C. for 18 h under nitrogen in the dark in a sealed microwave vessel. The resulting solution was cooled to room temperature and concentrated. The resulting brown semi-solid was sonicated in a small volume (10 mL) of a 1:1 mixture of tetrahydrofuran and acetone. Petrol (10 mL) was added to the solution, producing a precipitate. The solid was isolated via vacuum filtration and washed with diethyl ether (3×3 mL) to afford 3-(2-(dimethyl(propyl)-λ⁴-azaneyl)ethyl-1,1,2,2-d₄)-4-hydroxy-1H-indole iodide (35 mg, 29%) as a solid. LCMS (+ve mode): m/z=251.25 [M+H]⁺; ¹H NMR (D20, 300 MHz) δ 7.17 (s, 1H, ArH), 7.08 (m, 2H, 2×ArH), 6.56 (dd, 1H, J=5.3, 3.0 Hz, ArH), 3.31 (m, 2H, CH₂), 3.12 (s, 6H, 2×CH₃), 1.83 (m, 2H, CH₂), 0.95 (t, 3H, J=7.3 Hz, CH₃).

Synthesis of 4-Acetoxy-3-(2-(tris(methyl-d₃)λ⁴-azaneyl)ethyl-1,1,2,2-d₄)-1H-indole iodide (compound 10)

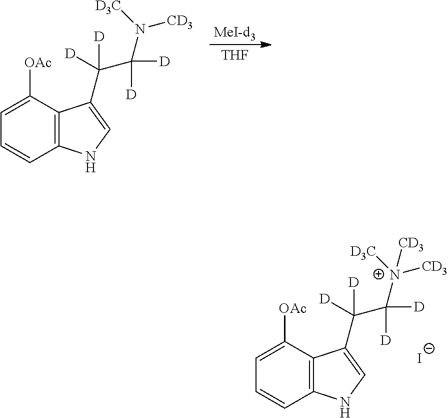 [SMILES: [2H]C([2H])([2H])N(C([2H])([2H])[2H])C([2H])([2H])C([2H])([2H])c1c[nH]c2cccc(OC(C)=O)c12 ; "MeI-d3" ; [2H]C([2H])(c1c[nH]c2cccc(OC(C)=O)c12)C([2H])([2H])[N+](C([2H])([2H])[2H])(C([2H])([2H])[2H])C([2H])([2H])[2H] ; [I-] ; "THF"]

To a solution of 3-(2-(bis(methyl-d₃)amino)ethyl-1,1,2,2-d₄)-1H-indol-4-yl acetate [CAS No: 2684212-32-4] (97 mg, 0.40 mmol, 1.0 equiv.) in anhydrous tetrahydrofuran (15 mL) was added iodomethane-d₃ (7.0 g, 3.0 mL, 48.2 mmol, 121.5 equiv.). The mixture was heated at 66° C. for 18 h under nitrogen in the dark in a sealed microwave vessel. The resulting suspension was cooled to room temperature. The precipitate was isolated via vacuum filtration and washed with diethyl ether (3×3 mL) to afford 4-acetoxy-3-(2-(tris(methyl-d₃)-2⁴-azaneyl)ethyl-1,1,2,2-d₄)-1H-indole iodide (110 mg, 69%) as a solid. LCMS (+ve mode): m/z=274.20 [M+H]⁺; ¹H NMR (D20, 300 MHz) δ 7.45 (d, 1H, J=8.2 Hz, ArH), 7.31 (s, 1H, ArH), 7.25 (t, 1H, J=7.9 Hz, ArH), 6.89 (d, 1H, J=7.7 Hz, ArH), 2.46 (s, 3H, CH₃).

Synthesis of 4-Hydroxy-3-(2-(tris(methyl-d₃)λ⁴-azaneyl)ethyl-1,1,2,2-d₄)-1H-indole iodide (compound 4)

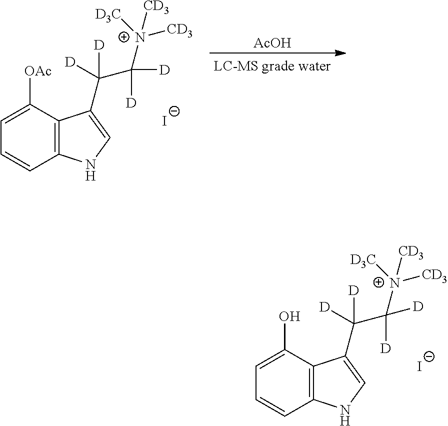 [SMILES: "AcOH" ; [2H]C([2H])(c1c[nH]c2cccc(OC(C)=O)c12)C([2H])([2H])[N+](C([2H])([2H])[2H])(C([2H])([2H])[2H])C([2H])([2H])[2H] ; [I-] ; [2H]C([2H])(c1c[nH]c2cccc(O)c12)C([2H])([2H])[N+](C([2H])([2H])[2H])(C([2H])([2H])[2H])C([2H])([2H])[2H] ; [I-]]

To a suspension of 4-acetoxy-3-(2-(tris(methyl-d₃)-λ⁴-azaneyl)ethyl-1,1,2,2-d₄)-1H-indole iodide (95 mg, 0.24 mmol, 1.0 equiv.) in LC-MS grade water (1.5 mL) was added acetic acid (5.3 g, 5.0 mL, 87.3 mmol, 368.4 equiv.). The mixture was heated at 100° C. for 18 h under nitrogen in the dark in a sealed microwave vessel. The resulting solution was cooled to room temperature and concentrated. The resulting brown semi-solid was sonicated in a small volume (4 mL) of acetonitrile. Petrol (2 mL) and diethyl ether were added to the solution, producing a precipitate. The solid was isolated via vacuum filtration and washed with petrol (3×3 mL) to afford 4-hydroxy-3-(2-(tris(methyl-d₃)-λ⁴-azaneyl)ethyl-1,1,2,2-d₄)-1H-indole iodide (50 mg, 59%) as a solid. LCMS (+ve mode): m/z=232.25 [M+H]⁺; ¹H NMR (D20, 300 MHz) δ 7.16 (s, 1H, ArH), 7.08 (m, 2H, 2×ArH), 6.56 (dd, 1H, J=5.6, 2.7 Hz, ArH).

Synthesis of 4-Acetoxy-3-(2-(methylbis(methyl-d₃)-λ⁴-azaneyl)ethyl-1,1,2,2-d₄)-1H-indole iodide (compound 11)

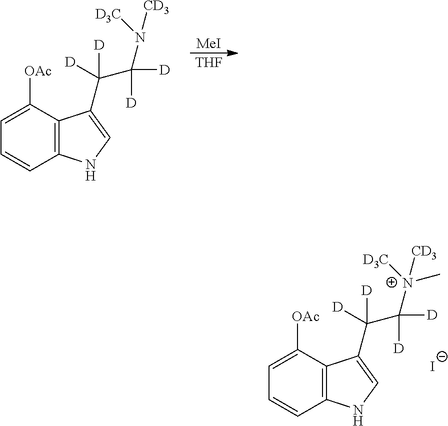 [SMILES: "MeI" ; [2H]C([2H])([2H])N(C([2H])([2H])[2H])C([2H])([2H])C([2H])([2H])c1c[nH]c2cccc(OC(C)=O)c12 ; [2H]C([2H])(c1c[nH]c2cccc(OC(C)=O)c12)C([2H])([2H])[N+](C)(C([2H])([2H])[2H])C([2H])([2H])[2H] ; [I-] ; "THF"]

To a solution of 3-(2-(bis(methyl-d₃)amino)ethyl-1,1,2,2-d₄)-1H-indol-4-yl acetate (97 mg, 0.40 mmol, 1.0 equiv.) in anhydrous tetrahydrofuran (15 mL) was added methyl iodide (6.8 g, 3.0 mL, 48.2 mmol, 121.4 equiv.). The mixture was heated at 66° C. for 18 h under nitrogen in the dark in a sealed microwave vessel. The resulting suspension was cooled to room temperature. The precipitate was isolated via vacuum filtration and washed with diethyl ether (3×3 mL) to afford 4-acetoxy-3-(2-(methylbis(methyl-d₃)-λ⁴-azaneyl)ethyl-1,1,2,2-d₄)-1H-indole iodide (94 mg, 59%) as a beige solid. LCMS (+ve mode): m/z=271.25 [M+H]⁺; ¹H NMR (D20, 300 MHz) δ 7.45 (d, 1H, J=8.2 Hz, ArH), 7.32 (s, 1H, ArH), 7.24 (t, 1H, J=7.8 Hz, ArH), 6.89 (d, 1H, J=7.7 Hz, ArH), 3.18 (s, 3H, CH₃), 2.46 (s, 3H, CH₃).

Synthesis of 4-Hydroxy-3-(2-(methylbis(methyl-d₃)-λ⁴-azaneyl)ethyl-1,1,2,2-d₄)-1H-indole iodide (compound 5)

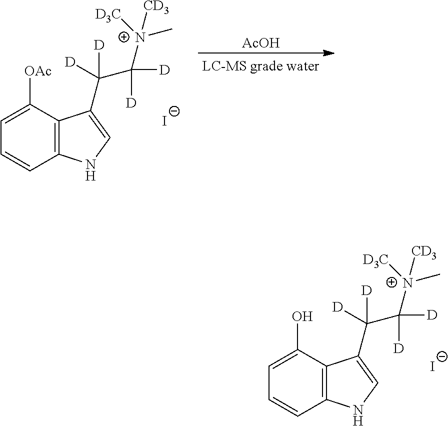 [SMILES: "AcOH" ; [2H]C([2H])(c1c[nH]c2cccc(OC(C)=O)c12)C([2H])([2H])[N+](C)(C([2H])([2H])[2H])C([2H])([2H])[2H] ; [I-] ; [2H]C([2H])(c1c[nH]c2cccc(O)c12)C([2H])([2H])[N+](C)(C([2H])([2H])[2H])C([2H])([2H])[2H] ; [I-]]

To a suspension of 4-acetoxy-3-(2-(methylbis(methyl-d₃)-λ⁴-azaneyl)ethyl-1,1,2,2-d₄)-1H-indole iodide (80 mg, 0.20 mmol, 1.0 equiv.) in LC-MS grade water (1.5 mL) was added acetic acid (5.3 g, 5.0 mL, 87.3 mmol, 434.3 equiv.). The mixture was heated at 100° C. for 18 h under nitrogen in the dark in a sealed microwave vessel. The resulting solution was cooled to room temperature and concentrated. The resulting brown semi-solid was sonicated in a small volume (4 mL) of a 1:1 mixture of tetrahydrofuran and acetone. Petrol (10 mL) was added to the solution, producing a precipitate. The solid was isolated via vacuum filtration and washed with petrol (3×3 mL) to afford 4-hydroxy-3-(2-(methylbis(methyl-d₃)-λ⁴-azaneyl)ethyl-1,1,2,2-d₄)-1H-indole iodide (30 mg, 42%) as a solid. LCMS (+ve mode): m/z=229.25 [M+H]⁺; ¹H NMR (D20, 300 MHz) δ 7.17 (s, 1H, ArH), 7.08 (m, 2H, 2×ArH), 6.56 (dd, 1H, J=5.6, 2.8 Hz, ArH), 3.18 (s, 3H, CH₃).

Synthesis of 4-Acetoxy-3-(2-(ethylbis(methyl-d₃)-λ⁴-azaneyl)ethyl-1,1,2,2-d₄)-1H-indole iodide (compound 12)

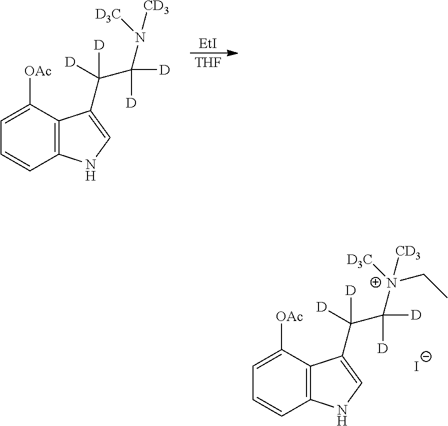 [SMILES: "EtI" ; [2H]C([2H])([2H])N(C([2H])([2H])[2H])C([2H])([2H])C([2H])([2H])c1c[nH]c2cccc(OC(C)=O)c12 ; [2H]C([2H])(c1c[nH]c2cccc(OC(C)=O)c12)C([2H])([2H])[N+](CC)(C([2H])([2H])[2H])C([2H])([2H])[2H] ; [I-] ; "THF"]

To a solution of 3-(2-(bis(methyl-d₃)amino)ethyl-1,1,2,2-d₄)-1H-indol-4-yl acetate (97 mg, 0.40 mmol, 1.0 equiv.) in anhydrous tetrahydrofuran (15 mL) was added ethyl iodide (5.80 g, 3.0 mL, 37.3 mmol, 94.0 equiv.). The mixture was heated at 66° C. for 18 h under nitrogen in the dark in a sealed microwave vessel. The resulting suspension was cooled to room temperature. The precipitate was isolated via vacuum filtration and washed with diethyl ether (3×3 mL) to afford 4-acetoxy-3-(2-(ethylbis(methyl-d₃)-λ⁴-azaneyl)ethyl-1,1,2,2-d₄)-1H-indole iodide (110 mg, 67%) as a solid. LCMS (+ve mode): m/z=285.25 [M+H]⁺; ¹H NMR (D20, 300 MHz) δ 7.45 (d, 1H, J=7.8 Hz, ArH), 7.31 (s, 1H, ArH), 7.25 (t, 1H, J=8.0 Hz, ArH), 6.89 (d, 1H, J=7.7 Hz, ArH), 3.43 (q, 2H, J=7.3 Hz, CH₂), 2.46 (s, 3H, CH₃), 1.35 (t, 3H, J=7.2 Hz, CH₃).

Synthesis of 3-(2-(Ethylbis(methyl-d₃)-λ⁴-azaneyl)ethyl-1,1,2,2-d₄)-4-hydroxy-1H-indole iodide (compound 6)

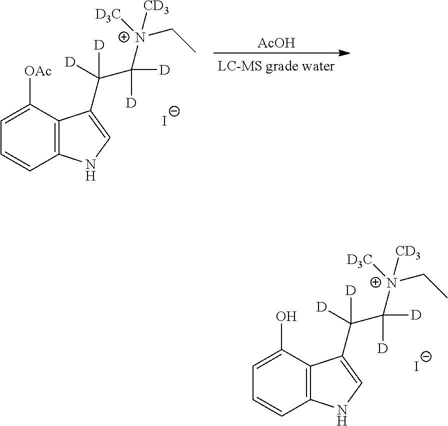 [SMILES: "AcOH" ; [2H]C([2H])(c1c[nH]c2cccc(OC(C)=O)c12)C([2H])([2H])[N+](CC)(C([2H])([2H])[2H])C([2H])([2H])[2H] ; [I-] ; [2H]C([2H])(c1c[nH]c2cccc(O)c12)C([2H])([2H])[N+](CC)(C([2H])([2H])[2H])C([2H])([2H])[2H] ; [I-]]

To a suspension of 4-acetoxy-3-(2-(ethylbis(methyl-d₃)-λ⁴-azaneyl)ethyl-1,1,2,2-d₄)-1H-indole iodide (95 mg, 0.23 mmol, 1.0 equiv.) in LC-MS grade water (1.5 mL) was added acetic acid (5.3 g, 5.0 mL, 87.3 mmol, 379.6 equiv.). The mixture was heated at 100° C. for 18 h under nitrogen in the dark in a sealed microwave vessel. The resulting solution was cooled to room temperature and concentrated. The resulting brown semi-solid was sonicated in a small volume (4 mL) of a 1:1 mixture of tetrahydrofuran and acetone. Petrol (10 mL) was added to the solution, producing a precipitate. The solid was isolated via vacuum filtration and washed with petrol (3×3 mL) to afford 3-(2-(ethylbis(methyl-d₃)-λ⁴-azaneyl)ethyl-1,1,2,2-d₄)-4-hydroxy-1H-indole iodide (55 mg, 65%) as a solid. LCMS (+ve mode): m/z=243.25 [M+H]⁺; ¹H NMR (D20, 300 MHz) δ 7.16 (s, 1H, ArH), 7.08 (m, 2H, 2×ArH), 6.55 (dd, 1H, J=5.6, 2.8 Hz, ArH), 3.43 (q, 2H, J=7.3 Hz, CH₂), 1.37 (t, 3H, J=7.3 Hz, CH₃).

### Example 2

Evaluation of Metabolic Stability in Human Liver Microsomes Microsomal Assay: Human liver microsomes (20 mg/mL) are obtained. B-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to from 12.5 μM to 50 μM in acetonitrile (CAN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 pL aliquot of the 12.5 μM to 50 μM dilution of the test compound is added to the microsomes and the mixture is pre-warmed for 10 min. Reactions are initiated by addition of pre-warmed NADPH solution. The final reaction volume is 0.5 mL and contains 4.0 mg/mL human liver microsomes, 0.25 μM test compound, 2 mM NADPH in 0.1 M potassium phosphate buffer, pH 7.4, and 3 mM MgCl₂. The reaction mixtures are incubated at 37° C., and 50 pL aliquots are removed at 0, 5, 10, 20, and 30 min or other suitable interval and added to shallow-well 96-well plates containing 50 pL of ice-cold CAN (acetonitrile) with internal standard to stop the reactions. The plates are stored at 4° C. for 20 minutes after which 100 pL of water is added to the wells of the plate before centrifugation to pellet precipitated proteins.

Supernatants are transferred to another 96-well plate and analysed for amounts of the test compound remaining by LC-MS/MS using, for example, an Applied Bio-systems API 4000® mass spectrometer. The same procedure is followed for the non-deuterated counterparts of the test compounds and for the positive control, 7-ethoxy coumarin (1 μM). Testing can be done in triplicate.

Data analysis: The in vitro T½s for the test compounds is calculated from the slopes of the linear regression of % test compound remaining (In) vs incubation time.

In vitro T½=0.693/k (Eqn.1)

where k=-[slope of linear regression of % parent remaining (In) vs incubation time. The apparent intrinsic clearance is calculated using the following equation:

CLᵢₙₜ(mL/min/kg)=(0.693/in vitroT)(Incubation Volume/mgof microsomes)(45mg microsomes/gram of liver)(20gmof liver/kg b.w.)(Eqn.2)

Data analysis can be performed using Microsoft Excel® Software.

In these experiments, values reflecting an equal to or more than a 15% increase in half-life are considered to be significant if the apparent intrinsic clearance ratio (deuterated compound/aeruginascin, 4-OH-TMT, 4-OAc-TMT) is greater than 1.15 or less than 0.85.

TABLE 2
Metabolic stability in human liver microsomes of representative deuterated compounds

| | | CLᵢₙₜ | | | |
|---|---|---|---|---|---|
| Compound | | (μL/min/mg | | t_{1/2} | |
| number | Compound name | protein) | SE CLᵢₙₜ | (min) | Comment |
| — | 4-OH-Trimethyltryptamine | 6.81 | 1.29 | 204 | |
| | iodide (reference compound A) | | | | |
| — | 4-OH-Dimethylethyltryptamine | 5.41 | 2.34 | 256 | |
| | iodide (reference compound B) | | | | |
| — | 4-OH-Dimethylpropyltryptamine | Not | ND | ND | |
| | iodide (reference compound C) | determined | | | |
| | | (ND) | | | |
| 1 | 4-Hydroxy-3-(2-(trimethyl- | 5.60 | 3.06 | 247 | |
| | λ⁴-azaneyl)ethyl-1,1,2,2-d₄)- | | | | |
| | 1H-indole iodide (compound 1) | | | | |
| 2 | 3-(2-(Ethyldimethyl-λ⁴- | 2.81 | 3.37 | 494 | |
| | azaneyl)ethyl-1,1,2,2-d₄)-4- | | | | |
| | hydroxy-1H-indole iodide (compound 2) | | | | |
| 3 | 3-(2-(Dimethyl(propyl)-λ⁴- | 1.42 | 3.82 | 973 | |
| | azaneyl)ethyl-1,1,2,2-d₄)-4- | | | | |
| | hydroxy-1H-indole iodide (compound 3) | | | | |
| 4 | 4-Hydroxy-3-(2-(tris(methyl-d₃)-λ⁴- | 0.401 | 2.44 | 3460 | |
| | azaneyl)ethyl-1,1,2,2-d₄)- | | | | |
| | 1H-indole iodide (compound 4) | | | | |
| 5 | 4-Hydroxy-3-(2-(methylbis(methyl-d₃)-λ⁴- | 0.228 | 2.81 | 6070 | |
| | azaneyl)ethyl-1,1,2,2-d₄)- | | | | |
| | 1H-indole iodide (compound 5) | | | | |
| 6 | 3-(2-(Ethylbis(methyl-d₃)-λ⁴- | 4.38 | 2.92 | 317 | |
| | azaneyl)ethyl-1,1,2,2-d₄)- | | | | |
| | 4-hydroxy-1H-indole iodide (compound 6) | | | | |
| — | 4-OAc-Trimethyltryptamine iodide | 28.3 | 2.48 | 48.9 | Minus cofactor control low (51% of 0 min). |
| | (reference compound D) | | | | Possible chemical instability or |
| | | | | | non-cofactor dependent enzymatic degradation |
| — | 4-OAc-Dimethylethyltryptamine | 46.4 | 1.55 | 29.9 | Minus cofactor control low (38% of 0 min). |
| | iodide (reference compound E) | | | | Possible chemical instability or |
| | | | | | non-cofactor dependent enzymatic degradation |
| — | 4-OAc-Dimethylpropyltryptamine | 62.7 | 3.78 | 22.1 | Minus cofactor control low (27% of 0 min). |
| | iodide (reference compound F) | | | | Possible chemical instability or |
| | | | | | non-cofactor dependent enzymatic degradation |
| 7 | 4-Acetoxy-3-(2-(trimethyl-λ⁴- | 29.7 | 1.68 | 46.7 | Minus cofactor control low (52% of 0 min). |
| | azaneyl)ethyl-1,1,2,2-d₄)- | | | | Possible chemical instability or |
| | 1H indole iodide (compound 7) | | | | non-cofactor dependent enzymatic degradation |
| 8 | 4-Acetoxy-3-(2-(ethyldimethyl-λ⁴- | 48.4 | 1.99 | 28.6 | Minus cofactor control low (37% of 0 min). |
| | azaneyl)ethyl-1,1,2,2-d₄)- | | | | Possible chemical instability or |
| | 1H-indole iodide (compound 8) | | | | non-cofactor dependent enzymatic degradation |
| 9 | 4-Acetoxy-3-(2-(dimethyl(propyl)-λ⁴- | 74.3 | 2.80 | 18.7 | Minus cofactor control low (20% of 0 min). |
| | azaneyl)ethyl-1,1,2,2-d₄)- | | | | Possible chemical instability or |
| | 1H-indole iodide (compound 9) | | | | non-cofactor dependent enzymatic degradation |
| 10 | 4-Acetoxy-3-(2-(tris(methyl-d₃)-λ⁴- | 29.8 | 5.30 | 46.5 | Minus cofactor control low (44% of 0 min). |
| | azaneyl)ethyl-1,1,2,2-d₄)- | | | | Possible chemical instability or |
| | 1H-indole iodide (compound 10) | | | | non-cofactor dependent enzymatic degradation |
| 11 | 4-Acetoxy-3-(2-(methylbis(methyl-d₃)-λ⁴- | 29.0 | 3.35 | 47.8 | Minus cofactor control low (51% of 0 min). |
| | azaneyl)ethyl-1,1,2,2-d₄)- | | | | Possible chemical instability or |
| | 1H-indole iodide (compound 11) | | | | non-cofactor dependent enzymatic degradation |
| 12 | 4-Acetoxy-3-(2-(ethylbis(methyl-d₃)-λ⁴- | 41.4 | 2.33 | 33.5 | Minus cofactor control low (40% of 0 min). |
| | azaneyl)ethyl-1,1,2,2-d₄)- | | | | Possible chemical instability or |
| | 1H-indole iodide (compound 12) | | | | non-cofactor dependent enzymatic degradation |
| — | dextromethorphan* (positive control) | 54.9 | 3.39 | 25.3 | |
| — | verapamil** (positive control) | 326.3 | 27.1 | 4.29 | 30 and 45 minute time points excluded |

*Average of n = 3 experiments
**Average of n = 3 experiments; 30- and 45-minute time points excluded because verapamil is metabolized very fast and is completely gone by 30 min. Thus, the 30 min and 45 min timepoints are not able to be measured.
ND = not determined.

Based on the results in Table 2, Compound 4-Hydroxy-3-(2-(trimethyl-λ⁴-azaneyl)ethyl-1,1,2,2-d₄)-1H-indole iodide (compound 1), 4-Hydroxy-3-(2-(tris(methyl-d₃)-λ⁴-azaneyl)ethyl-1,1,2,2-d₄)-1H-indole iodide (compound 4) and 4-Hydroxy-3-(2-(methylbis(methyl-d₃)λ⁴-azaneyl)ethyl-1,1,2,2-d₄)-1H-indole iodide (compound 5) exhibit significantly lower in vitro clearance than 4-OH-Trimethyltryptamine iodide (reference compound A). Compound 3-(2-(Ethyldimethyl-λ⁴-azaneyl)ethyl-1,1,2,2-d₄)-4-hydroxy-1H-indole iodide (compound 2) and 3-(2-(Ethylbis(methyl-d₃)-λ⁴-azaneyl)ethyl-1,1,2,2-d₄) hydroxy-1H-indole iodide (compound 6) exhibit significantly lower in vitro clearance than 4-OH-Dimethylethyltryptamine iodide (reference compound B). Compounds 7, 8, 9, 10, 11 and 12 exhibit differences in half-life and intrinsic clearance compared to the respective reference compounds.

### Example 3

Pharmacokinetics of Compounds Formula (2)

A pharmacokinetic (PK) study are performed in male Sprague-Dawley (SD) rats following intravenous (IV) and oral (PO) administration of aeruginascin, 4-OH TMT, 4-0Ac TMT, and deuterated analogs and derivatives thereof at doses 1 mg/kg (IV) and 10 (PO) mg/kg. Deuterated compounds of Formula (2), aeruginascin, 4-OH TMT, a 4-0Ac TMT, are measured in plasma.

A pharmacokinetic (PK) study is performed in three male Sprague-Dawley (SD) rats following intravenous (IV) and oral (PO) administrations of a test compound at 2 mg/kg and test compounds at 10 mg/kg, respectively and the test compounds are measured in plasma.

Test article is diluted 10% v/v DMSO, 40% v/v PEG-400, 50% v/v water. The test articles are administered in a dose volume of 2 mL/kg for intravenous (IV) and 5 mL/kg (PO) for oral routes of administration.

The dosing volumes are 5 mL/kg for IV and 10 mL/kg for PO. All aspects of this work including housing, experimentation, and animal disposal are performed in general accordance with the “Guide for the Care and Use of Laboratory Animals: Eighth Edition” (National Academies Press, Washington, D.C., 2011); and Suckow et al., Ed. The Laboratory Rat. 2nd Edition. Academic Press. New York. 2005. Animals have access to standard lab diet and autoclaved tap water ad libitum.

Blood aliquots (300 μL to 400 μL) are collected from jugular vein-catheterized rats into tubes coated with lithium heparin at various times. The tubes are mixed gently and kept on ice and then centrifuged at 2,500 rpm for 15 min at 4° C., within 1 h after collection. For animals in the control groups, blood is collected by cardiac puncture and the plasma is harvested and kept frozen at −70° C. until further analysis. Beaudoin et al., Bioanalytical method validation for the simultaneous determination of the test compound and/or metabolites thereof in rat plasma. Bioanalysis. 2016 8:111-22.

Plasma samples are processed using acetonitrile precipitation and analyzed by LC-MS/MS. A plasma calibration curve is generated with aliquots of drug-free plasma are spiked with the test compound at the specified concentration levels. The spiked plasma samples are processed together with the unknown plasma samples using the same procedure. The processed plasma samples are stored at −70° C. until receiving LC-MS/MS analysis, at which time peak areas are recorded, and the concentrations of the test compound in the unknown plasma samples are determined using the respective calibration curve. The reportable linear range of the assay is determined, along with the lower limit of quantitation (LLQ). Plots of plasma concentration of compound versus time are constructed. The pharmacokinetic parameters of compound after IV and PO dosing (AUCₗₐₛₜ, AUC_{INF}, T_{1/2}, Tₘₐₓ, and Cₘₐₓ) are obtained from the non-compartmental analysis (NCA) of the plasma data using WinNonlin® analysis software (WinNonlin® Certara L.P. Pharsight, St. Louis, Mo.).

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

1. A compound of Formula (2), 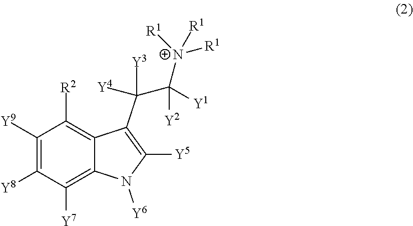
or a pharmaceutically acceptable salt thereof, wherein,
each R¹ is independently selected from CH₃, CH₂D, CHD₂, and CD₃;
R² is selected from an oxygen radical, a phosphate radical, a sulfate radical, and the moiety —O—C(═O)—R³ wherein R³ is selected from CH₃, CH₂D, CHD₂, and CD₃;
each of Y¹, Y², Y³, Y⁴, Y⁵, Y⁶, Y⁷, Y⁸, and Y⁹ is independently selected from hydrogen and deuterium; and
one or more of R¹, Y¹, Y², Y³, Y⁴, Y⁵, Y⁶, Y⁷, Y⁸, and Y⁹ comprises a deuterium.

2. The compound of claim 1, wherein the compound has the structure of Formula (2a), Formula (2b), Formula (2c), or Formula (2d):

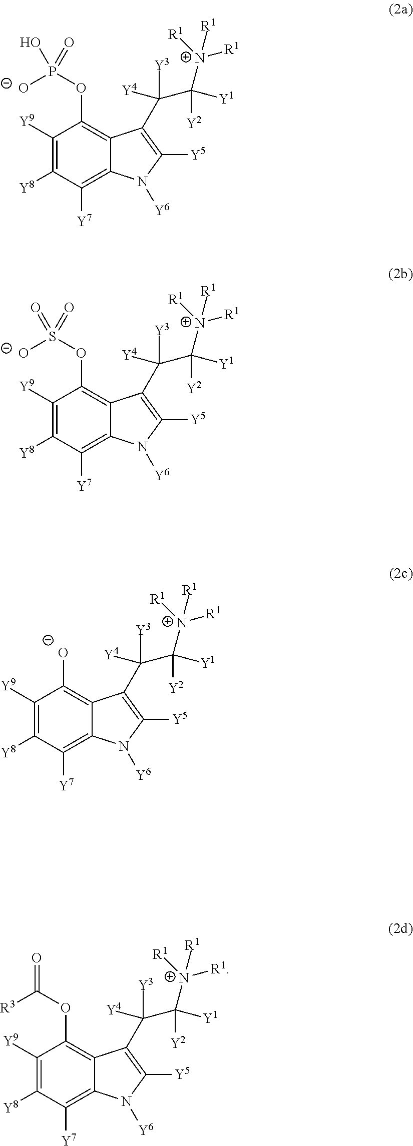

3. The compound of claim 2, wherein the compound has the structure of Formula (2a).

4. The compound of claim 2, wherein the compound has the structure of Formula (2b).

5. The compound of claim 2, wherein the compound has the structure of Formula (2c).

6. The compound of claim 2, wherein the compound has the structure of Formula (2d).

7-10. (canceled)

11. The compound of claim 1, wherein: (i) R² is —O—C(═O)—R³, and R³ is CH₃; (ii) R² is —O—C(═O)—R³, and R³ is CH₂D; (iii) R² is —O—C(═O)—R³, and R³ is CHD₂; or (iv) R² is —O—C(═O)—R³, and R³ is CD₃.

12-14. (canceled)

15. The compound of claim 1, wherein: (i) each of Y⁵, Y⁶, Y⁷, Y⁸, and Y⁹ is hydrogen; (ii) each of Y⁵, Y⁶, Y⁷, Y⁸, and Y⁹ is deuterium; (iii) one of Y⁵, Y⁶, Y⁷, Y⁸, and Y⁹ is hydrogen and each of the other of Y⁵, Y⁶, Y⁷, Y⁸, and Y⁹ is deuterium; (iv) two of Y⁵, Y⁶, Y⁷, Y⁸, and Y⁹ is hydrogen and each of the other of Y⁵, Y⁶, Y⁷, Y⁸, and Y⁹ is deuterium; (v) three of Y⁵, Y⁶, Y⁷, Y⁸, and Y⁹ is hydrogen and the other of Y⁵, Y⁶, Y⁷, Y⁸, and Y⁹ is deuterium; or (vi) four of Y⁵, Y⁶, Y⁷, Y⁸, and Y⁹ is hydrogen and the other of Y⁵, Y⁶, Y⁷, Y⁸, and Y⁹ is deuterium.

16-20. (canceled)

21. The compound of claim 11, wherein each R¹ is independently selected from CH₃, CH₂D, CHD₂, and CD₃.

22-24. (canceled)

25. The compound of claim 1, wherein each of Y¹, Y², Y and Y⁴ is deuterium.

26. The compound of claim 1, wherein each of Y¹ and Y² is deuterium, and each of Y³ and Y⁴ is hydrogen.

27. The compound of claim 1, wherein each of Y¹ and Y² is hydrogen, and each of Y³ and Y⁴ is deuterium.

28. The compound of claim 1, wherein each of Y¹, Y², and Y³ is hydrogen, and Y⁴ is deuterium.

29. The compound of claim 1, wherein each of Y² and Y³ is hydrogen, and each of Y¹ and Y⁴ is deuterium.

30. The compound of claim 1, wherein Y² is hydrogen, and each of Y¹, Y³ and Y⁴ is deuterium.

31. The compound of claim 1, wherein Y³ is hydrogen, and each of Y¹, Y² and Y⁴ is deuterium.

32. The compound of claim 1, wherein Y², Y³, and Y⁴ is hydrogen.

33. The compound of claim 1, wherein the compound is not selected from:
3-(2-(bis(methyl-d₃)amino)ethyl-1,1,2,2-d₄)-1H-indol-4-ol (3a);
3-(2-(dimethylamino)ethyl-1,1,2,2-d₄)-1H-indol-4-ol (3b);
3-(2-(bis(methyl-d₃)amino)ethyl)-1H-indol-4-ol (3c);
3-(2-(dimethylamino)ethyl-1,1,2,2-d₄)-1H-indol-4-yl acetate (3d);
3-(2-(bis(methyl-d₃)amino)ethyl-1,1,2,2-d₄)-1H-indol-4-yl acetate (3e);
3-(2-(bis(methyl-d₃)amino)ethyl)-1H-indol-4-yl acetate (3f);
3-(2-(dimethylamino)ethyl-1,1,2,2-d₄)-1H-indol-4-yl hydrogen phosphate (3g);
3-(2-(bis(methyl-d₃)amino)ethyl-1,1,2,2-d₄)-1H-indol-4-yl hydrogen phosphate (3h); and
3-(2-(bis(methyl-d₃)amino)ethyl)-1H-indol-4-yl hydrogen phosphate (3i).

34. A pharmaceutical composition comprising the compound of claim 1, or a pharmaceutically acceptable salt thereof.

35-36. (canceled)

37. A method treating a disease in a patient comprising administering to a patient in need of such treatment a therapeutically effective amount of the compound of claim 1, a pharmaceutically acceptable salt thereof, or a pharmaceutical composition thereof, wherein the disease is selected from a psychological disease, an inflammatory disease, pain, a brain disease, and a developmental disease.

38-40. (canceled)
